(19) United States
(12) Patent Application Publication
(10) Pub. No.: US 2024/0316057 A1
(43) Pub. Date: Sep. 26, 2024

(54) PULSATILE RELEASE CAFFEINE FORMULATION

(71) Applicant: GALVENTA AG, Gallen (TR)

(72) Inventors: Firat Yerlikaya, Çankaya (TR); Aslihan ArslanÇ, Çankaya (TR)

(73) Assignee: GALVENTA AG, Gallen (TR)

(21) Appl. No.: 18/271,088

(22) PCT Filed: Jan. 14, 2022
(86) PCT No.: PCT/EP2022/050768

§ 371 Date: Jul. 6, 2023

(30) Foreign Application Priority Data
Jan. 15, 2021 (EP) 21151905.3

(51) Int. Cl. A61K 31/522; A61K 9/00; A61K 9/20; A61K 9/28; A61K 47/12; A61K 47/26; A61K 47/36; A61K 47/38

(52) U.S. Cl. CPC A61K 31/522; A61K 9/0053; A61K 9/0095; A61K 9/2009; A61K 9/2013; A61K 9/2027; A61K 9/2031; A61K 9/28; A61K 47/12; A61K 47/26; A61K 47/36; A61K 47/38

(57) ABSTRACT

The present invention relates to a pulsatile release formulation comprising a nootropic agent and a release controlling polymeric system, as well as its therapeutic and non-therapeutic uses. The instant formulations are particularly useful in the therapy of morning depression and/or wake-up difficulties in Delayed Sleep Phase Syndrome, Seasonal Affective Disorders, Narcolepsy, Sleep Deprivation, Major Depression, Idiopathic Insomnia, or Sedative-induced Hang-overs.

### CROSS-REFERENCE TO RELATED APPLICATIONS

This application is the U.S. National Stage application, pursuant to 35 U.S.C. § 371, of PCT International Patent Application No. PCT/EP2022/050768, filed Jan. 14, 2022, designating the United States and published in English, which claims priority under 35 U.S.C. §§ 119 and 365 to European Patent Application No. 21151905.3, filed Jan. 15, 2021. The contents of each of the aforementioned applications are incorporated herein by reference in their entirety.

### FIELD OF THE INVENTION

The present invention relates to a pulsatile release formulation comprising a nootropic agent and a release controlling polymeric system, as well as its therapeutic and non-therapeutic uses. The instant formulations are particularly useful in treating wake-up difficulties, which are highly prevalent in the healthy population, but particularly in patients suffering from neurological, psychiatric, circadian and sleep-related disorders.

### BACKGROUND OF THE INVENTION

The World Health Organization (WHO) defines stimulants as substances which have an increasing, accelerating or improving effect on nerve activity. Today, stimulants are administered in various galenic forms. Liquid dosage forms include solutions, emulsions or suspensions, drops or syrups. There are also granulates, capsules and tablets for oral use and semi-solid preparations such as creams, ointments, gels, lotions, suspensions, emulsions and pastes for cutaneous use. There are also preparations for parenteral use. These include dosage forms administered via intravenous (IV), intramuscular (IM) or subcutaneous (SC) routes or via inhalation. Transdermal therapeutic systems (TDDS), such as therapeutic patches, also belong to the group of parenteral preparations. The available dosage forms of vitalizing substances are characterized in that after the administration of the stimulants an immediate release from the galenic formulation takes place and the absorption process of the respective substance into the organism (for example, via the blood circulation) starts immediately. The same applies to sedative substances. However, this is not desirable in every case.

The biggest problem of instantaneous drug release is probably that at the time of awakening a vitalization of the body would have to take place, however no immediate prior intake of a stimulant can take place, because the body is inactive during sleep. Therefore, for the majority of society, the morning awakening process is one of the more unpleasant inevitabilities of everyday life. A person's circadian rhythm, also known as sleep-wake rhythm, does not usually follow an externally determined or desired clock, but is subject to natural endogenous clocks (also referred to as circadian clock). These are predetermined to a certain degree by genetic components but can be influenced by external environmental stimuli such as sunlight and seasons, as well as by somatic, psychological and other stimuli. The deliberate influence on circadian rhythms, however, has so far often proved difficult if not impossible. The inner clock also changes as a person grows up. The inner clocks of adolescents are almost all slowed down during puberty. Many people maintain their inner rhythm, which is slower than the 24-hour rhythm, and struggle to get up early in the morning for the rest of their lives. But even habitual early risers cannot always rely on their inner clock and are also dependent on an external control organ: Alarm clocks in a wide variety of designs provide a remedy by awakening the body abruptly from its sleep state through acoustic, optical and/or sensory signals.

The homeostatic bodily functions are all reduced in the sleep state. Body temperature, pulse, respiratory rate and blood pressure are lowered, and brain activity is altered. The body is in a kind of “stand-by” mode, going through deeper and more superficial sleep phases during sleep. In the first hours of sleep, the melatonin level reaches its highest level and then slowly decreases. In addition, the body releases the wake-up hormone cortisol, which, along with dozens of other messengers and signals, initiates the state of wakefulness. However, if the body is torn from a deep sleep phase by artificial stimuli, without the waking hormones and signals being able to unfold their effect, the awakened person feels restless and exhausted, a feeling that often accompanies him throughout the day. The triggering of alarm signals is programmed in an external alarm clock via the time setting, independent of the sleep phase of the person concerned. It may be regularly ripped from the deep sleep phase by the alarm. The alarm clock is therefore a necessary evil that considerably impairs the quality of life of many people.

So-called bio-alarm clocks or sleep trackers, which are supposed to determine the individual sleep cycles by sensory measurements, are known. They take advantage of the fact that the movements of the body allegedly correlate with the respective sleep phases. These movements can be recorded via a sensor attached to the arm, for example, or via the acceleration sensors built into a smartphone, wherein these movements can be recorded and evaluated via a suitable software program. In the latter case, the smartphone is placed on the mattress, for example near the pillow. Bio-alarm clocks thus determine the phases of superficial sleep and trigger the morning alarm in such a user-defined time window. However, such sensory measurements are subject to considerable uncertainty and their effectiveness has not been sufficiently scientifically proven. Even if such a bio-alarm clock could locate the superficial sleep phases, there would still be no sufficiently reliable wake-up possibility, because morning appointments, timetables etc. do not depend on the individual sleep phase. In addition, although awakening from a deep sleep phase is more difficult than awakening from a superficial sleep phase, each awakening process is accompanied by an unpleasant feeling of sleepiness, also referred to as “sleep inertia”, regardless of the specific sleep phase from which the person wakes up.

Upon awakening, the majority of people take a vitalizing substance immediately afterwards. The vitalizing substance can be a coffee, which contains the stimulating substance caffeine as its main active ingredient. Caffeine is the most commonly consumed pharmacologically active substance in the world. It enables many people to eliminate symptoms of sleep inertia (after having been awake for some time) and at the same time increase their mental performance and physical endurance.

As known to the skilled person, caffeine in combination with sleep (so-called coffee naps) as a measure against fatigue is more effective than any of these two measures alone. It was shown that a person who was tired while driving and who consumed a drink with high caffeine content and then went to sleep to be woken up by the previously ingested caffeine after about half an hour was then involved in significantly fewer accidents than the control group. However, the measures supported by this finding cannot be applied to waking up in the morning, as it would require the person to take the caffeine half an hour before the desired wake-up time. In this sense, a dose of caffeine that could be released during sleep would be more effective than taking the same dose of caffeine after waking up. This is due to the occupancy rate of the adenosine receptors, which is elevated in the waking state and thus the caffeine has to compete with the endogenous adenosine. While asleep, on the other hand, the receptors remain largely unbound, and the caffeine therefore has a higher apparent association constant to the receptors, in other words it can bind more strongly due to the lack of competition. If the caffeine could still be administered during sleep, the required dose could be reduced to achieve the desired wake-up effect. Studies have shown that caffeine influences the inner clock (the so-called circadian clock). It is known that when caffeine is consumed in the evening, the inner clock shifts forward, i.e. the consumer gets tired later than his inner clock actually pretends. This property of caffeine (and also other substances; especially adenosine receptor agonists and antagonists) can also be used to reset the internal clock, i.e. to turn an evening type (late riser) into a morning type (early riser). This is also supported by the fact that caffeine leads to a cortisol release, which is known for its waking properties. In addition to caffeine, there are other nootropic substances that many people take every day. In the broadest sense, nootropics as understood herein are substances that have a beneficial effect on mental performance. Nootropics may include drugs, dietary supplements or other substances. Nootropics may also be referred to as smart drugs or psycholeptics. Well-known nootropics are in particular the substances caffeine and its derivatives, amphetamine and its derivatives, gingko, ginseng and amino acids available without prescription, as well as the active ingredient modafinil. Other examples also include piracetam and methylphenidate.

Despite their widespread use, there are no nootropic agents that have a time-controlled pulsatile release kinetics. Availability of such nootropic agent and/or formulation would allow to perform a time-controlled, precisely definable and variably adjustable vitalization and/or sedation of the body in different situations and for different purposes.

Patent application US 2002/0132003 discloses a certain method to deliver an orally administrable pharmaceutical, containing an outer layer of time delayed coating and a core of active substance to assist in the waking process of the human sleep cycle.

Patent application US 2016/0128943 discloses a certain oral formulation comprising a first component providing for immediate or rapid release of caffeine, and a second component providing for extended release of caffeine, as well as a method of administering the said formulation.

Patent application CH711042A2 discloses a certain preparation for a time-controlled pharmacological vitalization and/or sedation of the human or animal body.

Patent application CA232757 discloses certain method to deliver an orally administrable pharmaceutical, containing an outer layer of time delayed coating and a core of active substance to assist in the waking process of the human sleep cycle.

U.S. Pat. No. 5,242,941 as well as U.S. Pat. No. 5,707,652 disclose certain method for treating circadian rhythm disorders.

Patent application US 2010/0151023 discloses certain time-release energizing supplement.

Patent application WO 2002/072033 discloses certain chronotherapeutic pharmaceutical formulation.

Patent application WO 1995/003043 discloses a certain dosage form comprising a sustained release melatonin formulation.

Document US 2014/0370113 discloses certain pharmaceutical formulation comprising hydrocortisone and its use in the treatment of conditions that would benefit from a delayed release of hydrocortisone.

Document US 2001/038863 discloses certain pharmaceuticals with predetermined activity profile.

Bott et al. (Bott et al., Aliment. Pharmacol. Ther., vol 20, no. 3, 14 Jul. 2004, pages 347-353, DOI: 10.1111/j.1365-2036.2004.02033.x) discloses in vivo evaluation of a novel pH- and time-based multiunit colonic drug delivery system.

Document WO 2016/177834 discloses certain preparation for the pharmaceological vitalization, sedation or a chronological combination thereof by means of time-controlled release kinetics.

Chang et al. (Chang et al, “Polymethacrylates” in “Handbook of Pharmaceutical Excipients”, January 2009, Pharmaceutical Press, UK, pages 525-533) discloses relevant theory with respect to the polymeric systems disclosed herein.

### SUMMARY OF THE INVENTION

It was an object of the present invention to provide a pulsatile release formulation comprising nootropic agent and a release controlling polymeric system.

It is desirable that once orally administered, the pulsatile release formulation of the invention while passing through the gastrointestinal (GI) tract and being exposed to different pH values varying from strongly acidic in the stomach to slightly basic in the colon, does not significantly release the nootropic agent in the initial (acidic-to-neutral) parts of the GI tract, creating 4-8 hours of lag-time of nootropic agent release after the administration. It is further desirable that once the formulation passes to the further parts of the GI tract's (neutral-to-basic) the release-controlling polymeric system immediately dissolves to provide a rapid release of the nootropic agent. It is further desirable that rapid release of the nootropic agent is observed within 5-10 hours after the administration. The present inventors have surprisingly found that a copolymer or copolymers of anionic and basic methacrylate monomers, as described herein, can be used to provide a specific release profile, as described herein.

The technical problem is solved as described herein and as characterized in the claims.

The invention can be summarized in the following embodiments.

In a first embodiment the present invention relates to a pulsatile release formulation comprising a nootropic agent and a release-controlling polymeric system of the present invention, wherein the release controlling polymeric system comprises:
(a) a copolymer of methacrylic acid and methyl methacrylate in a molar ratio of 1:2.5 to 1:1.5;
(b) a copolymer of methacrylic acid and methyl methacrylate in a molar ratio of 1:1.4 to 1:0.5; and
(c) a copolymer of ethyl acrylate, methyl methacrylate and trimethylammonioethyl methacrylate chloride, wherein ethyl acrylate and methyl methacrylate are present in the molar ratio of 1:2.5 to 1:1.5 and wherein ethyl acrylate and trimethylammonioethyl methacrylate chloride are present in the molar ratio of 1:0.25 to 1:0.05; wherein (a) and (b) are present in a weight/weight ratio of between 2 to 6, wherein (c) and (b) are present in a weight/weight ratio of between 0.5 to 2, and wherein the nootropic agent's release is controlled by the release-controlling polymeric system.

In a particular embodiment the present invention relates to a pulsatile release formulation comprising a nootropic agent and a release-controlling polymeric system of the present invention, wherein (a) has a weight average molecular weight of between 120 000 Da and 130 000 Da, and/or wherein (b) has a weight average molecular weight of between 120 000 Da and 130 000 Da, and/or wherein (c) has a weight average molecular weight of between 30 000 Da and 34 000 Da.

In a further particular embodiment the present invention relates to a pulsatile release formulation comprising a nootropic agent and a release-controlling polymeric system of the present invention, wherein (a) has a weight average molecular weight of about 125 000 Da, and/or wherein (b) has a weight average molecular weight of about 125 000 Da, and/or wherein (c) has a weight average molecular weight of about 32 000 Da.

In again a further particular embodiment the present invention relates to a pulsatile release formulation comprising a nootropic agent and a release-controlling polymeric system of the present invention, wherein (a) is a copolymer of methacrylic acid and methyl methacrylate in a molar ratio of about 1:2.

In again a further particular embodiment the present invention relates to a pulsatile release formulation comprising a nootropic agent and a release-controlling polymeric system of the present invention, wherein (b) is a copolymer of methacrylic acid and methyl methacrylate in a molar ratio of about 1:1.

In again a further particular embodiment the present invention relates to a pulsatile release formulation comprising a nootropic agent and a release-controlling polymeric system of the present invention, wherein (a) and (b) are present in a weight/weight ratio of about 4.

In again a further particular embodiment the present invention relates to a pulsatile release formulation comprising a nootropic agent and a release-controlling polymeric system of the present invention, wherein the (c) and (b) are present in a weight/weight ratio of 1.0 to 1.5.

In again a further particular embodiment the present invention relates to a pulsatile release formulation comprising a nootropic agent and a release-controlling polymeric system of the present invention, wherein (a) is Eudragit® S and/or (b) is Eudragit® L and/or (c) is Eudragit® RS and/or (c) is Eudragit® RL.

In a second embodiment the present invention relates to a pulsatile release formulation comprising a nootropic agent and a release-controlling polymeric system of the present invention, wherein the release-controlling polymeric system comprises a copolymer of methacrylic acid, methyl methacrylate and methyl acrylate,
wherein the molar ratio of methacrylic acid to the sum of methyl methacrylate and methyl acrylate is between 0.05 to 0.15,
wherein the molar ratio of methyl acrylate and methyl methacrylate is from 2.0 to 2.8, and
wherein the nootropic agent's release is controlled by the release controlling polymeric system.

In a further particular embodiment the present invention relates to a pulsatile release formulation comprising a nootropic agent and a release-controlling polymeric system of the present invention, wherein the weight average molecular weight of the copolymer is between 260 000 Da and 300 000 Da.

In again a further particular embodiment the present invention relates to a pulsatile release formulation comprising a nootropic agent and a release-controlling polymeric system of the present invention, wherein the weight average molecular weight of the copolymer is about 280 000 Da.

In again a further particular embodiment the present invention relates to a pulsatile release formulation comprising a nootropic agent and a release-controlling polymeric system of the present invention, wherein the molar ratio of methacrylic acid to the sum of methyl methacrylate and methyl acrylate is about 0.10, and wherein the molar ratio of methyl acrylate and methyl methacrylate is about 2.3.

In again a further particular embodiment the present invention relates to a pulsatile release formulation comprising a nootropic agent and a release-controlling polymeric system of the present invention, wherein the copolymer is Eudraguard® biotic.

In a particular embodiment, the present invention relates to a pulsatile-release formulation of the present invention, as defined herein, wherein the nootropic agent is an adenosine receptor agonist.

In a further particular embodiment, the present invention relates to a pulsatile-release formulation of the present invention, as defined herein, wherein the adenosine receptor agonist is xanthin derivative, in particular theophylline or a pharmaceutically acceptable salt thereof, theobromine or a pharmaceutically acceptable salt thereof, paraxanthine or a pharmaceutically acceptable salt thereof, or caffeine or a pharmaceutically acceptable salt thereof.

In again a further particular embodiment, the present invention relates to a pulsatile-release formulation of the present invention, as defined herein, wherein the nootropic agent is caffeine.

In again a further particular embodiment, the present invention relates to a pulsatile-release formulation of the present invention, wherein the nootropic agent is a sympathomimetic, in particular ephedrine or a pharmaceutically acceptable salt thereof, amphetamine or a pharmaceutically acceptable salt thereof, phenylephrine or a pharmaceutically acceptable salt thereof, pseudoephedrine or a pharmaceutically acceptable salt thereof, or etilefrine or a pharmaceutically acceptable salt thereof.

In again a further particular embodiment, the present invention relates to a pulsatile-release formulation of the present invention, wherein the nootropic agent is piracetam or a pharmaceutically acceptable salt thereof.

In again a further particular embodiment, the present invention relates to a pulsatile-release formulation of the present invention, wherein the nootropic agent is phenylpiracetam or a pharmaceutically acceptable salt thereof.

In again a further particular embodiment, the present invention relates to a pulsatile-release formulation of the present invention, wherein the nootropic agent is modafinil or a pharmaceutically acceptable salt thereof.

In again a further particular embodiment, the present invention relates to a pulsatile-release formulation of the present invention, wherein the nootropic agent is armodafinil or a pharmaceutically acceptable salt thereof.

In again a further particular embodiment, the present invention relates to a pulsatile-release formulation of the present invention, wherein the nootropic agent is methylphenidate or a pharmaceutically acceptable salt thereof.

In again a further particular embodiment, the present invention relates to a pulsatile-release formulation of the present invention, wherein the nootropic agent is a serotonin and noradrenalin reuptake inhibitor, in particular venlafaxine or a pharmaceutically acceptable salt thereof, desvenlafaxine or a pharmaceutically acceptable salt thereof, or duloxetine or a pharmaceutically acceptable salt thereof, or wherein the nootropic agent is a selective serotonin reuptake inhibitor, in particular fluoxetine or a pharmaceutically acceptable salt thereof, sertraline or a pharmaceutically acceptable salt thereof, paroxetine or a pharmaceutically acceptable salt thereof, citalopram or a pharmaceutically acceptable salt thereof, escitalopram or a pharmaceutically acceptable salt thereof, or fluvoxamine or a pharmaceutically acceptable salt thereof.

In again a further particular embodiment, the present invention relates to a pulsatile-release formulation of the present invention, wherein the ratio of the nootropic agent's weight to the total solid content's weight of the pulsatile-release formulation is between 10:1 to 1:100.

In again a further particular embodiment, the present invention relates to a pulsatile-release formulation of the present invention, wherein the ratio of the weight of the release-controlling polymeric system to the weight of the rest of the pulsatile-release formulation is between 1:20 to 5:1.

In a particular embodiment, the present invention relates to a pulsatile-release formulation of the present invention, further comprising a sedative substance.

In a further particular embodiment, the present invention relates to a pulsatile-release formulation of the present invention, wherein the sedative substance is melatonin or a pharmaceutically acceptable salt thereof.

In again a further particular embodiment, the present invention relates to a pulsatile-release formulation of the present invention, wherein the sedative substance is an antihistaminic, in particular diphenhydramine or a pharmaceutically acceptable salt thereof, dimenhydrinate or a pharmaceutically acceptable salt thereof, or dimetindene or a pharmaceutically acceptable salt thereof.

In again a further particular embodiment, the present invention relates to a pulsatile-release formulation of the present invention, wherein the sedative substance is an antipsychotic, in particular quetiapine or a pharmaceutically acceptable salt thereof, levomepromazine or a pharmaceutically acceptable salt thereof, or olanzapine or a pharmaceutically acceptable salt thereof.

In again a further particular embodiment, the present invention relates to a pulsatile-release formulation of the present invention, wherein the sedative substance is trazodone or a pharmaceutically acceptable salt thereof.

In again a further particular embodiment, the present invention relates to a pulsatile-release formulation of the present invention, wherein the sedative substance is mirtazapine or a pharmaceutically acceptable salt thereof.

In again a further particular embodiment, the present invention relates to a pulsatile-release formulation of the present invention, wherein the sedative substance is a z-drug, in particular zolpidem or a pharmaceutically acceptable salt thereof, zaleplon or a pharmaceutically acceptable salt thereof, or zolpidem or a pharmaceutically acceptable salt thereof.

In again a further particular embodiment, the present invention relates to a pulsatile-release formulation of the present invention, wherein the sedative substance is a benzodiazepine, in particular alprazolam or a pharmaceutically acceptable salt thereof, chlordiazepoxide or a pharmaceutically acceptable salt thereof, clonazepam or a pharmaceutically acceptable salt thereof, clorazepate or a pharmaceutically acceptable salt thereof, diazepam or a pharmaceutically acceptable salt thereof, estazolam or a pharmaceutically acceptable salt thereof, flurazepam (Dalmane) or a pharmaceutically acceptable salt thereof, lorazepam or a pharmaceutically acceptable salt thereof, midazolam or a pharmaceutically acceptable salt thereof, oxazepam or a pharmaceutically acceptable salt thereof, temazepam or a pharmaceutically acceptable salt thereof, triazolam or a pharmaceutically acceptable salt thereof, or quazepam or a pharmaceutically acceptable salt thereof.

In again a further particular embodiment, the present invention relates to a pulsatile-release formulation of the present invention, wherein the sedative substance is doxylamine or a pharmaceutically acceptable salt thereof.

In again a further particular embodiment, the present invention relates to a pulsatile-release formulation of the present invention, wherein the sedative substance is chlorphenamine or a pharmaceutically acceptable salt thereof.

In again a further particular embodiment, the present invention relates to a pulsatile-release formulation of the present invention, wherein the sedative substance comprises a phytochemical selected from Valeriana officinali, Humulus lupulus, Lavandula officinalis, Hypericum perforatum, Petasites hybridus, Melissa officinalis, Passiflora incarnata, and Choisya ternata.

In again a further particular embodiment, the present invention relates to a pulsatile-release formulation of the present invention, wherein the sedative substance comprises an amino acid selected from L-tryptophan, L-theanine, and glycine.

In again a further particular embodiment, the present invention relates to a pulsatile-release formulation of the present invention, wherein the sedative substance is niacin or a pharmaceutically acceptable salt thereof.

In again a further particular embodiment, the present invention relates to a pulsatile-release formulation of the present invention, wherein the sedative substance comprises magnesium salt.

In a further embodiment, the present invention relates to a pulsatile-release formulation comprising a nootropic agent for use in therapy.

In a further embodiment, the present invention relates to a pulsatile-release formulation of the present invention for use in therapy.

In a further embodiment, the present invention relates to a pulsatile-release formulation comprising a nootropic agent for use in therapy of morning depression and/or wake-up difficulties in Delayed Sleep Phase Syndrome, Seasonal Affective Disorders, Narcolepsy, Sleep Deprivation, Depression, Idiopathic Insomnia, or Sedative-induced Hangovers.

In a further embodiment, the present invention relates to a pulsatile-release formulation of the present invention for use in therapy of morning depression and/or wake-up difficulties in Delayed Sleep Phase Syndrome, Seasonal Affective Disorders, Narcolepsy, Sleep Deprivation, Depression, Idiopathic Insomnia, or Sedative-induced Hangovers.

In a particular embodiment, the present invention relates to a pulsatile-release formulation for use of the present invention, wherein the nootropic agent is caffeine, and wherein the strength of caffeine per dosage unit is between 10 mg and 1000 mg, more preferably between 40 mg and 320 mg, most preferably 80 mg or 160 mg.

In a further particular embodiment, the present invention relates to a pulsatile-release formulation for use of the present invention, that upon oral administration at bedtime has the ability to decrease subjective and objective signs of sleep inertia immediately upon morning awakening, indicated by improved scores in the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI).

In again a further particular embodiment, the present invention relates to a pulsatile-release formulation for use of the present invention, characterized by an in vitro release profile wherein not more than 25% of a nootropic agent is released during the first 5 hours and not less than 75% of a nootropic agent is released until the 9th hour, as measured by the analysis of cumulative dissolved amount of the nootropic agent upon incubation in the dissolution media having a pH of 1.2 for 2 hours, having a pH of 6.5 for 1 hour, having a pH of 6.8 for 2 hours and having a pH of 7.2 for 5 hours.

In again a further particular embodiment, the present invention relates to a pulsatile-release formulation for use of the present invention, characterized by an in vivo release profile in human subjects wherein the blood plasma concentration of caffeine reaches 5 μM not earlier than 4 hours following the administration of said pulsatile-release formulation.

In a further embodiment, the present invention relates to a use of the pulsatile-release formulation of the present invention for targeted shifting of the circadian sleep-wake rhythm.

In a particular embodiment, the present invention relates to the use of the present invention, wherein targeted shifting of the circadian sleep-wake rhythm is performed in the case of Jet Lag or Shift Work.

In a further particular embodiment, the present invention relates to the use of the present invention, wherein the nootropic agent is caffeine, and wherein the dose of caffeine is between 10 mg and 1000 mg, more preferably between 40 mg and 320 mg, most preferably 80 mg or 160 mg.

It is apparent to the skilled person that in order to prevent caffeine release during sleep period, the in vitro release should preferably show a lag time of 5 hours, there preferably should be no release until 5th hour, and preferably a burst release should occur to provide higher caffeine amount in the blood in the morning hours. The pulsatile release formulation comprising a nootropic agent and a release-controlling polymeric system of the first embodiment of the present invention differs from the formulation disclosed in US 2014/0370113 in (c):(b) weight/weight ratio. Formulation as disclosed in US 2014/0370113 has a weight/weight ratio of (c):(b) of 3.3, which is higher than the range of 0.5 to 2 of the present invention.

In the Reference Preparative Example 1 (also referred to as Reference Example 1) and Preparative Example 7 (also referred to as Example 7), the composition of the release controlling polymeric system is the same, except that the weight/weight ratio of polymers of (c):(b) differs, being 3.3 in the Reference Preparative example 1 and 1.25 (i.e., within the range of 0.5 to 2) for the Preparative Example 7. The remaining parameters are representative of the first embodiment of the present invention. The present inventors have shown that the formulation of the present invention (i.e., the formulation according to the first embodiment of the present invention, as described hereinabove) leads to a pulsatile burst release of nootropic agent (caffeine in the specific working example) after 5 hours, which is not the case for the formulation of the prior art (see FIG. 8A).

The pulsatile release formulation comprising a nootropic agent and a release-controlling polymeric system of the second embodiment of the present invention (in particular of the particular embodiment wherein the molar ratio of methacrylic acid to the sum of methyl methacrylate and methyl acrylate is about 0.10, and wherein the molar ratio of methyl acrylate and methyl methacrylate is about 2.3) differs from the formulation disclosed in Bott et al. in that the molar ratio of methyl acrylate and methyl methacrylate is different (2.6 in Eudragit FS 30D as used in Bott et al.). The Reference Preparative Example 2 (also referred to as Reference Example 2) is representative of formulation comprising Eudragit FS 30D used in Bott et al., while Preparative Example 12 (also referred to as Example 12) is representative of the second embodiment of the present invention, in particular wherein the molar ratio of methyl acrylate and methyl methacrylate is about 2.3. The present inventors have shown that the formulation of the present invention (i.e., the formulation according to the second embodiment of the present invention, as described hereinabove) leads to a pulsatile burst release of nootropic agent (caffeine in the specific working example) after 5 hours, which is not the case for the formulation of the prior art (see FIG. 8B).

### BRIEF DESCRIPTION OF FIGURES

FIG. 1: (A) Impact of the pulsatile release formulation of the present invention on the blood level of nootropic agent; (B) Illustration of Caffeine Pulsatile-Release Pellet; (C) In vitro release profile of the pulsatile-release formulation used in the clinical study.

FIG. 2: Illustration of the study procedures of the in vivo validation study (A) and the pharmacodynamic study (B). Timepoints of blood withdrawal are indicated as grey drops ( [symbol] ). Sleep episodes are highlighted as hatched areas. In the pharmacodynamic study, participants were kept awake until 3:00. In both studies, sleep was continuously recorded by polysomnography. Immediately after awakening, volunteers performed a 1-hour testing battery (referred to as “Testing”) to quantify behavioural, cognitive, emotional and physiological markers of sleep inertia. At 8:00, the participants were given a 1-hour nap opportunity, while the latency to fall asleep and the sleep profile were recorded with polysomnography.

FIG. 3: Evolution of the caffeine plasma concentration over time for the in vivo validation study (A) and the pharmacodynamic study (B). Black dots indicate mean caffeine plasma concentrations, error bars indicate standard errors (SEM). The horizontal dashed line at 5 μM indicates the threshold concentration of caffeine efficacy.

FIG. 4: Post-awakening (8:00-9:00) assessments of subjective state. ASIQ=Acute Sleep Inertia Questionnaire. CAQ=Caffeine Acute Questionnaire. PANAS-positive=positive affective scale. PANAS-negative=negative affective scale. *p<0.05**p<0.01 (Benjamini-Hochberg corrected).

FIG. 5: PVT reaction times (left) and number of lapses (right) at 2:30 and 7:00. Mean values (dots) and standard error of the mean (vertical lines) are shown. Grey lines indicate the placebo condition; black lines indicate the caffeine condition. ***p<0.001 (Benjamini-Hochberg corrected).

FIG. 6: Salivary Cortisol Awakening Response (CAR). Mean salivary cortisol concentration (dots) and standard error of the mean (vertical lines) are shown. Grey lines indicate the placebo condition; black lines indicate the caffeine condition. *p<0.05 (Benjamini-Hochberg corrected).

FIG. 7: Nighttime sleep (3:00-7:00; panel A) and nap variables (8:00-9:00; panel B) are shown. SOL, sleep onset latency; N1-3, sleep stages N1-3; REM, rapid-eye movement sleep; *p<0.05 (Benjamini-Hochberg corrected).

FIG. 8: Comparison of the in vitro release characteristic for (A) (Preparative) Example 7 and Reference (Preparative) Example 1; (B) (Preparative) Example 12 and Reference (Preparative) Example 2;

### DETAILED DESCRIPTION OF THE INVENTION

The formulations of the present invention will be described in the following. It is to be understood that all possible combinations of the following features are also envisaged.

The present invention relates to a pulsatile-release formulation comprising a nootropic agent and a release-controlling polymeric system. As understood herein, the nootropic agent's release is controlled by the release-controlling polymeric system. According to the present invention, the release-controlling polymeric system comprises at least one copolymer. The at least one copolymer as defined herein is a copolymer of at least methacrylic acid and methyl methacrylate. It is understood herein that the copolymer as defined herein is obtainable by copolymerization of a reaction mixture comprising at least methacrylic acid and methyl methacrylate. The presence of other compounds that can be polymerised in the reaction mixture is not excluded according to the present invention. Accordingly, the copolymer of the present invention may be obtainable by copolymerization of methacrylic acid, methyl methacrylate and further compound(s) capable of (co)polymerization.

The structure of a polymer or of a copolymer, as known to the skilled person, may also be defined through the presence of certain repeating unit(s). Accordingly, the copolymer of the present invention comprises repeating unit(s) according to formula:

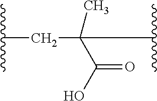

which is incorporated into the copolymer through polymerization of methacrylic acid. The copolymer of the present invention further comprises repeating unit(s) according to formula:

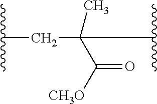

which is incorporated into the copolymer through polymerization of methyl methacrylate. As understood herein, the copolymer of the present invention may comprise further repeating units.

In certain embodiments of the present invention, the copolymer of the present invention comprises no further repeating units, beyond the repeating units as defined above. Thus, in certain embodiments the copolymer of the present invention may be a copolymer of methacrylic acid and methyl methacrylate.

The copolymer of the present invention may be in certain embodiments a copolymer of methacrylic acid and methyl methacrylate. In certain embodiments, the methacrylic acid and the methyl methacrylate are in the molar ratio of 1:2.5 to 1:1.5, preferably 1:2.3 to 1:1.7, more preferably 1:2.1 to 1:1.9. Most preferably, the methacrylic acid and the methyl methacrylate are in the molar ratio of about 1:2, even more preferably in the molar ratio of 1:2.

The copolymer of the present invention as defined herein is preferably characterized by the weight average molecular weight of between 100 000 Da and 150 000 kDa. More preferably, the copolymer of the present invention is characterised by the weight average molecular weight of between 110 000 Da and 140 000 Da. Even more preferably, the copolymer of the present invention is characterised by the weight average molecular weight of between 120 000 Da and 130 000 Da. As understood herein, the weight average molecular weight is preferably determined by using the SEC MALS determination method.

The copolymer of the present invention may be in certain embodiments a copolymer of methacrylic acid and methyl methacrylate wherein the methacrylic acid and the methyl methacrylate are in the molar ratio of about 1:2, wherein the copolymer of the present invention is characterised by the weight average molecular weight of between 120 000 Da and 130 000 Da. Thus, in certain embodiments, the polymer of the present invention is Eudragit® S, as commercially available from Evonik.

The copolymer of the present invention may be in certain embodiments a copolymer of methacrylic acid and methyl methacrylate. In certain embodiments, the methacrylic acid and the methyl methacrylate are in the molar ratio of 1:1.4 to 1:0.5, preferably 1:1.2 to 1:0.8, more preferably 1:1.1 to 1:0.9. Most preferably, the methacrylic acid and the methyl methacrylate are in the molar ratio of about 1:1, even more preferably in the molar ratio of 1:1.

The copolymer of the present invention as defined herein is preferably characterized by the weight average molecular weight of between 100 000 Da and 150 000 kDa. More preferably, the copolymer of the present invention is characterised by the weight average molecular weight of between 110 000 Da and 140 000 Da. Even more preferably, the copolymer of the present invention is characterised by the weight average molecular weight of between 120 000 Da and 130 000 Da. As understood herein, the weight average molecular weight is preferably determined by using the SEC MALS determination method.

The copolymer of the present invention may be in certain embodiments a copolymer of methacrylic acid and methyl methacrylate wherein the methacrylic acid and the methyl methacrylate are in the molar ratio of about 1:1, wherein the copolymer of the present invention is characterised by the weight average molecular weight of between 120 000 Da and 130 000 Da. Thus, in certain embodiments, the polymer of the present invention is Eudragit® L, as commercially available from Evonik.

In certain embodiments of the present invention, the release-controlling polymeric system comprises more than one copolymer. In certain embodiments, the release-controlling polymeric system comprises copolymer (a) as defined below and a copolymer (b), as defined below. Copolymer (a) is a copolymer of methacrylic acid and methyl methacrylate in a molar ratio of 1:2.5 to 1:1.5, preferably 1:2.3 to 1:1.7, more preferably 1:2.1 to 1:1.9, even more preferably the methacrylic acid and the methyl methacrylate are in the molar ratio of about 1:2, most preferably of 1:2. Copolymer (b) is a copolymer of methacrylic acid and methyl methacrylate in a molar ratio of 1:1.4 to 1:0.5, preferably 1:1.2 to 1:0.8, more preferably 1:1.1 to 1:0.9, even more preferably. the methacrylic acid and the methyl methacrylate are in the molar ratio of about 1:1, most preferably in the molar ration of 1:1.

As understood herein, each copolymer (a) and/or copolymer (b) as defined herein is/are preferably characterized by the weight average molecular weight of between 100 000 Da and 150 000 kDa, more preferably of between 110 000 Da and 140 000 Da, even more preferably of between 120 000 Da and 130 000 Da. As understood herein, the weight average molecular weight is preferably determined by using the SEC MALS determination method.

In certain embodiments, the release-controlling polymeric system comprises a copolymer (a) as defined below and a copolymer (b), as defined below. A copolymer (a) is a copolymer of methacrylic acid and methyl methacrylate in a molar ratio of 1:2, characterized by the weight average molecular weight of between 120 000 Da and 130 000 Da. A copolymer (b) is a copolymer of methacrylic acid and methyl methacrylate in a molar ratio of 1:1, characterized by the weight average molecular weight of between 120 000 Da and 130 000 Da, As understood herein (a) and (b) are present in a weight/weight ratio of between 2 to 6, preferably in a weight/weight ratio of 3 to 5, more preferably in a weight/weight ratio of about 4, most preferably in a weight/weight ratio of 4. Preferably, in certain embodiments (a) is Eudragit® S as commercially available from Evonik, and/or (b) is Eudragit® L, as commercially available from Evonik.

In certain embodiments of the present invention the release-controlling polymeric system in addition to at least one copolymer of at least methacrylic acid and methyl methacrylate, may include further polymers. In one embodiment of the present invention the release-controlling polymeric system of the present invention further comprises a copolymer of ethyl acrylate, methyl methacrylate and trimethylammonioethyl methacrylate chloride. Preferably, as defined herein ethyl acrylate and methyl methacrylate are present in the molar ratio of 1:2.5 to 1:1.5, more preferably in the molar ratio of 1:2.3 to 1:1.7, even more preferably in the molar ratio of 1:2.1 to 1:1.9. Even more preferably, ethyl acrylate and methyl methacrylate are present in the molar ratio of about 1:2, most preferably in the molar ratio of 1:2. Preferably as defined herein, ethyl acrylate and trimethylammonioethyl methacrylate chloride are present in the molar ratio of 1:0.25 to 1:0.05, more preferably of 1:0.2 to 1:0.1, even more preferably ethyl acrylate and trimethylammonioethyl methacrylate chloride are present in the molar ratio of about 1:0.15, most preferably in the molar ratio of 1:0.15. As defined herein, the copolymer of ethyl acrylate, methyl methacrylate and trimethylammonioethyl methacrylate chloride is characterized by a weight average molecular weight of between 30 000 Da and 34 000 Da, more preferably by a weight average molecular weight of about 32 000 Da.

In one embodiment, the release-controlling polymeric system of the present invention further comprises a copolymer of ethyl acrylate, methyl methacrylate and trimethylammonioethyl methacrylate chloride, wherein ethyl acrylate, methyl methacrylate and trimethylammonioethyl methacrylate chloride are present in the molar ratio of about 1:2:0.1, and wherein said copolymer is characterized by a weight average molecular weight of about 32 000 Da. Preferably, the said copolymer is Eudragit® RS, as commercially available from Evonik.

In one embodiment, the release-controlling polymeric system of the present invention further comprises a copolymer of ethyl acrylate, methyl methacrylate and trimethylammonioethyl methacrylate chloride, wherein ethyl acrylate, methyl methacrylate and trimethylammonioethyl methacrylate chloride are present in the molar ratio of about 1:2:0.2, and wherein said copolymer is characterized by a weight average molecular weight of about 32 000 Da. Preferably, the said copolymer is Eudragit® RL, as commercially available from Evonik.

In one embodiment of the present invention the release controlling polymeric system comprises copolymer (a) and copolymer (b) and copolymer (c), as defined below. Copolymer (a) is a copolymer of methacrylic acid and methyl methacrylate in a molar ratio of 1:2.5 to 1:1.5. preferably 1:2.3 to 1:1.7, more preferably 1:2.1 to 1:1.9, even more preferably the methacrylic acid and the methyl methacrylate are in the molar ratio of about 1:2, most preferably of 1:2. The copolymer (a) of the present invention is preferably characterised by the weight average molecular weight of between 120 000 Da and 130 000 Da. Copolymer (b) is a copolymer of methacrylic acid and methyl methacrylate in a molar ratio of 1:1.4 to 1:0.5, preferably 1:1.2 to 1:0.8, more preferably 1:1.1 to 1:0.9, even more preferably, the methacrylic acid and the methyl methacrylate are in the molar ratio of about 1:1, most preferably in the molar ratio of 1:1. The copolymer of the present invention is preferably characterised by the weight average molecular weight of between 120 000 Da and 130 000 Da. Copolymer (c) is a copolymer of ethyl acrylate, methyl methacrylate and trimethylammonioethyl methacrylate chloride, wherein ethyl acrylate and methyl methacrylate are present in the molar ratio of 1:2.5 to 1:1.5, more preferably in the molar ratio of 1:2.3 to 1:1.7, even more preferably in the molar ratio of 1:2.1 to 1:1.9, even more preferably, ethyl acrylate and methyl methacrylate are present in the molar ratio of about 1:2, most preferably in the molar ratio of 1:2, and wherein ethyl acrylate and trimethylammonioethyl methacrylate chloride are present in the molar ratio of 1:0.25 to 1:0.05, more preferably of 1:0.2 to 1:0.1, even more preferably ethyl acrylate and trimethylammonioethyl methacrylate chloride are present in the molar ratio of about 1:0.1, most preferably in the molar ratio of 1:0.1. The copolymer (c) is characterized by a weight average molecular weight of between 30 000 Da and 34 000 Da, more preferably by a weight average molecular weight of about 32 000 Da. As defined herein, (a) and (b) are present in a weight/weight ratio of between 2 to 6, preferably of 3 to 5, more preferably of about 4, even more preferably of 4. As further defined herein, (c) and (b) are present in a weight/weight ratio of between 0.5 to 2, more preferably of 1.0 to 1.5, even more preferably of about 1.25, most preferably of 1.25.

In one embodiment of the present invention the release controlling polymeric system comprises:
(a) a copolymer of methacrylic acid and methyl methacrylate in a molar ratio of 1:2.5 to 1:1.5;
(b) a copolymer of methacrylic acid and methyl methacrylate in a molar ratio of 1:1.4 to 1:0.5; and
(c) a copolymer of ethyl acrylate, methyl methacrylate and trimethylammonioethyl methacrylate chloride.

In the copolymer (c), ethyl acrylate and methyl methacrylate are present in the molar ratio of 1:2.5 to 1:1.5, and ethyl acrylate and trimethylammonioethyl methacrylate chloride are present in the molar ratio of 1:0.25 to 1:0.05. Preferably, (a) and (b) are present in a weight/weight ratio of between 2 to 6, further preferably (c) and (b) are present in a weight/weight ratio of between 0.5 to 2. As understood herein, the nootropic agent's release is controlled by the release-controlling polymeric system.

In a preferred embodiment of the present invention the release controlling polymeric system comprises:
(a) a copolymer of methacrylic acid and methyl methacrylate in a molar ratio of about 1:2;
(b) a copolymer of methacrylic acid and methyl methacrylate in a molar ratio of about 1:1; and
(c) a copolymer of ethyl acrylate, methyl methacrylate and trimethylammonioethyl methacrylate chloride. In the copolymer (c), ethyl acrylate and methyl methacrylate are present in the molar ratio of 1:2, and ethyl acrylate and trimethylammonioethyl methacrylate chloride are present in the molar ratio of 1:0.1. Preferably, (a) and (b) are present in a weight/weight ratio of between 2 to 6, more preferably 3 to 5, even more preferably about 4. Further preferably (c) and (b) are present in a weight/weight ratio of between 0.5 to 2, more preferably 1 to 1.5, even more preferably about 1.25. As understood herein, the nootropic agent's release is controlled by the release-controlling polymeric system. Preferably, the polymer (a) is characterized by a weight average molecular weight of between 120 000 Da and 130 000 Da. Preferably, in certain embodiments (a) is Eudragit® S as commercially available from Evonik, further preferably, the polymer (b) is characterized by a weight average molecular weight of between 120 000 Da and 130 000 Da. Preferably, the copolymer (b) is Eudragit® L, as commercially available from Evonik. Further preferably, the polymer (c) is characterized by a weight average molecular weight of between 30 000 Da and 34 000 Da, more preferably (c) is characterized by a weight average molecular weight of about 32 000 Da. Preferably, the copolymer (c) is Eudragit® RS or Eudragit® RL, as commercially available from Evonik.

Anionic methacrylate copolymers can be applied for pH-dependent release mechanism and basic methacrylate copolymers can be applied for pH-independent release mechanism. Anionic methacrylate copolymers (e.g., Eudragit® S and Eudragit® L) are useful as coating materials to prevent drug release in stomach and facilitate the release in intestinal or in the colonic regions. Basic methacrylate copolymers (e.g., Eudragit® RS and Eudragit® RL) have quaternary ammonium groups in the chloride salt form. The dissociation of these groups in aqueous media is responsible for the swellability and permeability of the polymers. Anionic methacrylate copolymers can be used alone or in combination with each other, because of their solubility in different pH values. For example, Eudragit® L dissolves at pH above 6 and Eudragit® S dissolves pH above 7. The combination of these two copolymers ensures that the coating begins to dissolve in the initial parts of the intestines, although thickness of coating prevents complete dissolution of the coating. Basic methacrylate copolymers may also be combined with anionic methacrylate copolymer polymers to achieve targeted release profiles. In the present invention, anionic and basic methacrylate copolymers are used in combination to provide a specific release profile.

In one embodiment of the present invention, the release-controlling polymeric system comprises a copolymer of methacrylic acid, methyl methacrylate and methyl acrylate. The molar ratio of methacrylic acid to the sum of methyl methacrylate and methyl acrylate is between 0.05 and 0.15, preferably between 0.07 and 0.13, more preferably between 0.09 and 1.11, even more preferably is about 0.1, most preferably is 0.1. Furthermore, the molar ratio of methyl acrylate and methyl methacrylate is between 2.0 and 2.8, preferably between 2.2 and 2.5, more preferably it is about 2.3, even more preferably it is 2.3. Preferably, a weight average molecular weight of the copolymer is between 260 000 Da and 300 000 Da, more preferably it is about 280 000 Da.

In a preferred embodiment, the release-controlling polymeric system comprises a copolymer of methacrylic acid, methyl methacrylate and methyl acrylate, wherein the molar ratio of methacrylic acid to the sum of methyl methacrylate and methyl acrylate is about 0.10, and wherein the molar ratio of methyl acrylate and methyl methacrylate is about 2.3, and wherein a weight average molecular weight of the copolymer is about 280 000 Da. Preferably, in certain embodiments of the present invention, the copolymer as defined herein is Eudraguard® biotic. It has been approved by the European Commission as a safe food additive and is used for targeted colonic delivery of food additives by providing pH-dependent release mechanism, since it dissolves at pH 7.0 or above.

As understood herein, the release of nootropic agent is controlled by the release-controlling polymeric system. In certain embodiments of the present invention, the nootropic agent may be enclosed by the release controlling polymeric system. In certain embodiments of the present invention, the nootropic agent may be encapsulated by the release-controlling polymeric system. However, these release mechanisms are not to be construed as limiting and any means of formulating the nootropic agent with the release controlling polymeric system are understood as being encompassed within the present invention.

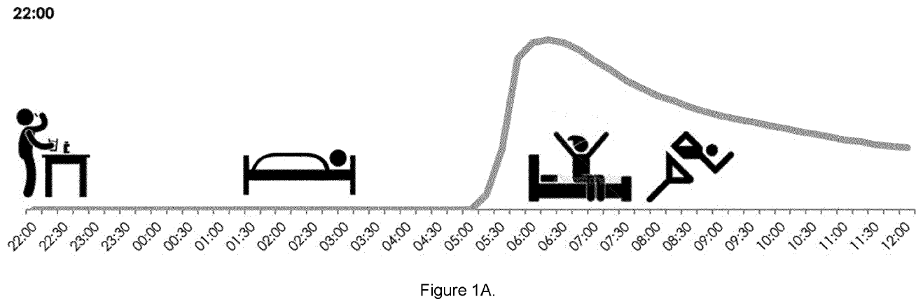

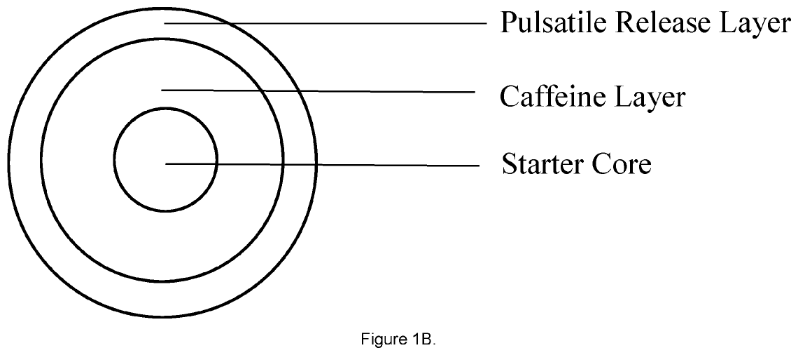

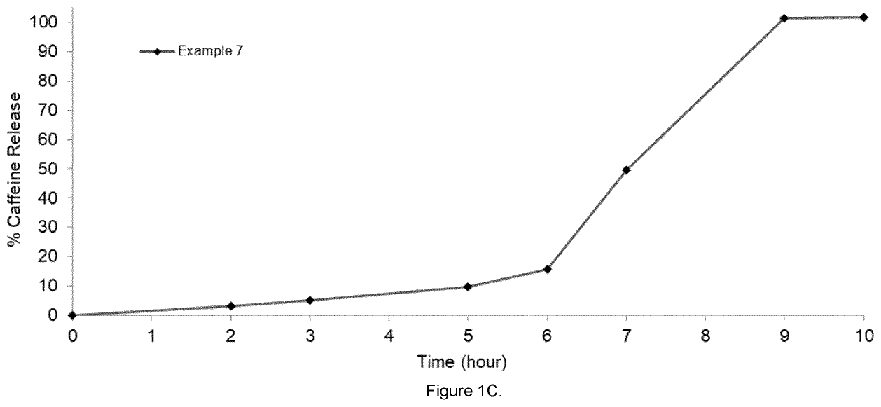

The present inventors have surprisingly found that the pulsatile release formulation of the present invention is suitable, after ingestion and expiry of a determinable period of time, for abruptly releasing the nootropic agent. FIG. 1A explains the pharmacological wake-up principle in more detail. The blood plasma concentration of a nootropic agent, such as caffeine, is plotted diagrammatically against time. The person concerned takes the nootropic agent-containing preparation orally at the previously determined time of administration, in this case at 22:00 hours (10 p.m.). As it can be seen from the FIG. 1A, the nootropic agent (herein caffeine) concentration in the blood plasma of the sleeping person does not increase during this time. The nootropic agent (caffeine) is formulated as a pulsatile release formulation and no significant amount of nootropic agent (caffeine) enters into the systemic circulation until the lag time has elapsed. For example, the formulation can be prepared so that the release of the nootropic agent does not occur until the early hours of the morning when the preparation is taken in the evening or, if the preparation is taken at 10 p.m., the nootropic agent (caffeine) is released from the dosage form at 5 a.m. for the first time. The preparation is then cracked, broken open, digested or dissolved to such an extent that the nootropic agent (caffeine) can be absorbed. Accordingly, between 5:00 a.m. and 6:00 a.m. a steep rise in the caffeine concentration in the person's blood plasma can be observed. As a result of the release of a nootropic agent (caffeine), the bodily functions that are reduced during sleep are activated, which accelerates the awakening process until the person becomes conscious at about 6:30 a.m.

The pulsatile release formulation of the present invention prevents the nootropic agent formulated therein from being released until the selected time. Therefore, the nootropic agent, for example caffeine, is released shortly before the wake-up time and then enters the bloodstream of the sleeping person. From this time on, the sleep of the person gradually becomes more superficial as the bodily functions are activated. Depending on the dose of the nootropic agent, for example caffeine, the awakening process is stimulated to a different extent. In one example, the person awakened at 6:25 a.m. does not exhibit any unpleasant wake-up symptoms thanks to the nootropic effect of the nootropic agent, herein caffeine.

As encompassed by the present invention, the release-controlling polymeric system of the present invention controls the release of a nootropic agent. However, as it would be apparent to a skilled person, the release controlling polymeric system as defined herein can be used to control the release of any substance, not only of a nootropic agent. Sedatives with time-controlled pulsatile release of active substances can also be formulated as described herein for the nootropics. Typically, the person in question is exposed to stress, e.g., from an upcoming examination, or an important meeting, on the following day. As a result, the person is already under stress on the previous evening, in this case before taking the sedative-containing preparation. Nervousness and stress sensitivity are psychosomatic, which is why confidence in a substance with an unmistakable effect at the desired time can make a significant contribution to the person's well-being. As a result, the person will also be able to sleep better and will feel more relaxed and rested. Due to this form of inhibited release of the active substance, the person is optimally served with a minimum dose of sedative. Instead of having to take sedative substances for a longer period of time until the decisive event, the person will experience the effective sedative effect by taking an optimally dosed preparation with time-controlled pulsatile release kinetics at the decisive point in time and will thus also be calmed in the preliminary phase of this event. The time-controlled pulsatile release kinetics of a sedative is advantageous in many respects. Taking a sedative before important appointments is often difficult because the person is on the road shortly before and/or still has a lot of things to do. The intake of conventional sedating preparations and the associated planning of the ideal point in time for taking them, which can only be determined imprecisely anyway, are perceived as an additional burden in a stressed state. A preparation with time-controlled pulsatile release of the active ingredient has no such disadvantage. The person will be calmer overall and, in particular, sedated during the period of the event important to him or her.

As it is apparent to the skilled person, the drug release from a galenic formulation is normally subject to strong intra- and interindividual differences (pharmacokinetic and pharmacodynamic differences). This is due to the fact that anatomical and physiological factors such as metabolism, gastrointestinal peristalsis, degree of saturation (food intake), enzyme activity, acid balance, etc. vary greatly between and within individuals. This means that for a punctual, precisely time-controlled pulsatile release of the active ingredient, herein the nootropic agent, the galenic form of the preparation must not depend on external parameters, but must be released in every human subject according to substantially the same kinetics, regardless of the degree of saturation, pH conditions, enzymatic activity, etc., the dose of the active ingredient at a predefined point in time. This is not achieved by single unit formulations such as tablets, capsules, dragees, etc., since differences often arise between the individual doses during the production of the tablets, capsules, etc., which then results in variable release kinetics. Preferably, a dose must be distributed into as many units as possible, so that an interindividual variability between the individual units can be balanced. This means that the total release of the active substance follows a Gaussian distribution when the dose is distributed over many small particles and is therefore better suited for a time-controlled up effect.

Within the scope of the present invention, any nootropic agent can be used. Preferably, the nootropic agent is an adenosine receptor agonist. As understood to the skilled person, adenosine receptors are G-protein coupled receptors, in particular four adenosine receptors as present in humans and known as A₁, A₂ₐ, A_{2b} and A₃. As understood herein, the adenosine receptor agonist is a molecule that binds at least one of the adenosine receptors in a way that leads to similar response to that caused by adenosine.

It is to be understood that while the working examples that support the present invention are performed with caffeine being the nootropic agent, the skilled person would recognize that the pulsatile release formulation of the present invention would work with any nootropic agent, in particular with any nootropic agent as described herein. Thus, it is apparent to the skilled person that any data presented for the release of caffeine from the pulsatile release formulation of the present invention wherein the nootropic agent is caffeine would support the formulations of the invention with nootropic agent beyond caffeine.

The neuromodulator adenosine is known to the skilled person as a key regulator of deep sleep and adenosinergic neuromodulation may play an essential role in the manifestation of sleep inertia. Consistent with this view, the adenosine receptor antagonist, caffeine, is widely used to counteract sleep inertia. Besides attenuating sleepiness and deficits in vigilance, caffeine augments cardiovascular and respiratory functions and promotes the release of cortisol, a key hormone of the hypothalamic-pituitary-adrenal (HPA) axis. The HPA axis regulates several psycho-vegetative aspects of the wake-up process, including the cortisol awakening response (CAR) that reflects HPA axis function and may be associated with the propensity of sleep inertia.

Preferably within the scope of the present invention, the adenosine receptor agonist is xanthin derivative, in particular theophylline or a pharmaceutically acceptable salt thereof, theobromine or a pharmaceutically acceptable salt thereof, paraxanthine or a pharmaceutically acceptable salt thereof, or caffeine or a pharmaceutically acceptable salt thereof.

In certain embodiments of the present invention, the adenosine receptor agonist is theophylline or a pharmaceutically acceptable salt thereof. The chemical name of theophylline is 1,3-dimethylxanthine, and its chemical structure is shown in formula below:

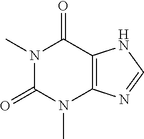

In certain embodiments of the present invention, the adenosine receptor agonist is theobromine or a pharmaceutically acceptable salt thereof. Theobromine is a very slightly water-soluble, white or almost white powder. The chemical name of theobromine is 3,7-dimethylxanthine, and its chemical structure is shown in formula below:

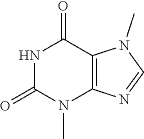

In certain embodiments of the present invention, the adenosine receptor agonist is paraxanthine or a pharmaceutically acceptable salt thereof. The chemical name of paraxanthine is 1,7-dimethylxanthine, and its chemical structure is shown in the formula below:

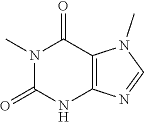

In certain embodiments of the present invention, the adenosine receptor agonist is caffeine or a pharmaceutically acceptable salt thereof. The chemical name of caffeine is 1,3,7-trimethylxanthine, and its chemical structure is shown in the formula below:

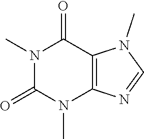

Caffeine is a white powder with a melting point between 234 and 239° C. It is sparingly soluble in water at room temperature. Caffeine can in anhydrous form, it may also include one water molecule to form the crystal monohydrate form. As understood herein, when referring to caffeine or a pharmaceutically acceptable form thereof, the caffeine monohydrate is also encompassed in this term.

Preferably, the adenosine receptor agonist is caffeine or a pharmaceutically acceptable salt thereof. Thus, in a preferred embodiment, the present invention relates to a pulsatile-release formulation of the present invention, wherein the nootropic agent is caffeine, as defined herein.

In certain embodiments of the present invention, the nootropic agent as comprised in the pulsatile release formulation of the present invention is a sympathomimetic. As defined herein, the term “sympathomimetic” or alternatively the term “sympathomimetic drug” relates to a compound or a formulation capable of mimicking the effects of endogenous agonists of the sympathetic nervous system. Without limitation to the scope of the present invention, it is acknowledged as known to the skilled person that endogenous agonists of the sympathetic nervous system include the catecholamines, including epinephrine (also referred to as adrenaline), norepinephrine (also referred to as noradrenaline) and dopamine, which are known to function as neurotransmitters and hormones. The examples of sympathomimetics, as defined herein, include ephedrine or a pharmaceutically acceptable salt thereof, amphetamine or a pharmaceutically acceptable salt thereof, phenylephrine or a pharmaceutically acceptable salt thereof, pseudoephedrine or a pharmaceutically acceptable salt thereof, and etilefrine or a pharmaceutically acceptable salt thereof. Thus, in certain embodiments of the present invention the nootropic agent is ephedrine or a pharmaceutically acceptable salt thereof, amphetamine or a pharmaceutically acceptable salt thereof, phenylephrine or a pharmaceutically acceptable salt thereof, pseudoephedrine or a pharmaceutically acceptable salt thereof, and etilefrine or a pharmaceutically acceptable salt thereof.

In another embodiment, the nootropic agent as comprised in the pulsatile release formulation of the present invention is piracetam or a pharmaceutically acceptable salt thereof. Piracetam is a compound marketed as cognitive enhances, the compound according to the formula:

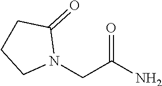

The pulsatile release formulation of the present invention may also comprise, instead of piracetam, its close analogue, phenylpiracetam, which is the compound according to the formula:

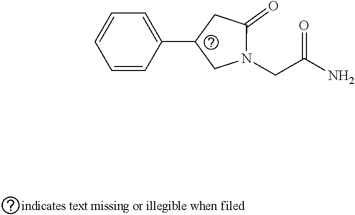 [SMILES: NC(=O)CN1CC(c2ccccc2)CC1=O]

Thus, in another embodiment, the present invention relates to a pulsatile-release formulation of the present invention, wherein the nootropic agent is phenylpiracetam or a pharmaceutically acceptable salt thereof.

In further embodiments, the nootropic agent as defined in the present invention and comprised within the pulsatile release formulation of the present invention can be selected from the group containing modafinil, armodafinil and methylphenidate. Thus, in one embodiment, the present invention relates to a pulsatile-release formulation of the present invention, wherein the nootropic agent is modafinil or a pharmaceutically acceptable salt thereof. In a further embodiment, the present invention relates to a pulsatile-release formulation of the present invention, wherein the nootropic agent is armodafinil or a pharmaceutically acceptable salt thereof. In again a further embodiment, the present invention relates to a pulsatile-release formulation of the present invention, wherein the nootropic agent is methylphenidate or a pharmaceutically acceptable salt thereof.

In certain embodiments, the present invention relates to a pulsatile-release formulation of the present invention, wherein the nootropic agent is a serotonin and noradrenalin reuptake inhibitor. As understood herein, the serotonin and noradrenalin reuptake inhibitor is a compound capable of blocking the serotonin transporter (SERT), and capable of blocking norepinephrine transporter (NET). Serotonin and noradrenalin reuptake inhibitors include venlafaxine, desvenlafaxine and duloxetine. Thus, in certain embodiments within the scope of the present invention, the nootropic agent is venlafaxine or a pharmaceutically acceptable salt thereof, desvenlafaxine or a pharmaceutically acceptable salt thereof, or duloxetine or a pharmaceutically acceptable salt thereof,

In certain embodiments, the present invention relates to a pulsatile-release formulation of the present invention, wherein the nootropic agent is a selective serotonin reuptake inhibitor. A selective serotonin reuptake inhibitor is defined herein as a compound capable of blocking the serotonin transporter, but not capable of blocking norepinephrine transporter or dopamine transporter. In other words, the selective serotonin reuptake inhibitor is defined herein as a compound with affinity for serotonin transporter at least 100-fold (preferably at least 1000-fold), higher than for norepinephrine transporter and dopamine transporter. The selective serotonin reuptake inhibitors include fluoxetine, sertraline, paroxetine, citalopram, escitalopram, and fluvoxamine. Therefore, the present invention relates to a pulsatile-release formulation of the present invention, wherein the nootropic agent fluoxetine or a pharmaceutically acceptable salt thereof, sertraline or a pharmaceutically acceptable salt thereof, paroxetine or a pharmaceutically acceptable salt thereof, citalopram or a pharmaceutically acceptable salt thereof, escitalopram or a pharmaceutically acceptable salt thereof, or fluvoxamine or a pharmaceutically acceptable salt thereof.

Possible preparations for the pulsatile release formulation of the present invention are any type of solid dosage form such as capsule or microcapsule systems, pellets, micropellets, nanopellets, tablets, mini-tablets, micro-tablet systems, dragees, lozenges, semi-solid dosage forms such as implants, transdermal delivery systems, and liquid dosage forms such as emulsions, suspensions, nasal sprays, oral sprays, The pulsatile release formulation of the present invention is administered preferably via the oral route. Most of the oral dosage forms such as pellets, micropellets, capsules, tablets, mini tablets, multilayer tablets, sachets, suspensions or combinations thereof, are suitable.

The most preferable dosage form for the pulsatile release formulation of the present invention is micropellets, wherein preferably an inert core such as sugar spheres or microcrystalline spheres are coated with one or more layers that comprise the nootropic agent and other layers that comprise release-controlling polymeric system of the present invention. Excipients such as solubilizers, binders, disintegrants, plasticizers, anti-tacking agents, glidants, anti-adherents, lubricants, film coating dispersions, and colorants may also be present either in the core or in any of the layers.

The pulsatile release formulation of the present invention is usually manufactured using the drug layering process, known to the skilled person. In the drug layering process, there are two possible methods to obtain the formulation of the invention, which are powder layering and liquid layering methods. In powder layering method, inert spheres are loaded into a fluid bed coater with a rotating disc at the bottom. A binder solution is sprayed into the fluid bed using one or more nozzles while a dry powder is directly sprayed into the fluid bed using some specific nozzles, concurrently. In liquid layering method, the nootropic agent and optionally the excipient(s) are dispersed or dissolved in a medium and then sprayed into the fluid bed through a spray apparatus (such as a Wurster tube, a tangential spray system or a top spray system). In both methods, there is a continuous inlet air that dries the liquid in the solution or in the dispersion.

Another common method to manufacture the pulsatile release formulation of the present invention is the direct pelletization process, where the nootropic agent is in the core of the micropellets with or without excipients such as fillers, diluents, solubilizers, binders, disintegrants, plasticizers, anti-tacking agents, glidants, anti-adherents, lubricants, film coating dispersions, and/or colorants. The direct pelletizing process for the production of cores with nootropic agent may include melt or wet granulation methods, followed by extrusion and/or spheronization processes. The direct pelletizing of the nootropic agent may be conducted using a rotor-fluidized bed process, which is the Glatt CPS™ technology (Complex Perfect Spheres Technology). The CPS™ technology works with a conically tilted rotating disc that allows for a directional particle motion. Powder mixture containing the nootropic agent and microcrystalline cellulose are loaded into the rotor-fluidized bed and wetted with a liquid (e.g. water with or without excipients). The pelletization process including wetting, drying and spheronization are performed in the same equipment. Melt and wet granulation processes, followed by extrusion and spheronization can also be used to obtain cores comprising the nootropic agent.

A single or a multilayer tablet form is also suitable for the present invention. In this approach, the core of a single layer tablet or the core of one of the layers of a multilayer tablet comprises the nootropic agent along with or without some excipients such as fillers, diluents, solubilizers, binders, disintegrants, plasticizers, anti-tacking agents, glidants, anti-adherents, lubricants, film coating dispersions, and colorants. The tablet cores are then coated with release-controlling polymers or copolymers.

In certain embodiments, the present invention relates to a pulsatile-release formulation of the present invention, wherein the ratio of the nootropic agent's weight to the total solid content's weight of the pulsatile-release formulation is between 10:1 to 1:100. The present invention further relates to a pulsatile-release formulation of the present invention, wherein the ratio of the weight of the release-controlling polymeric system to the weight of the rest of the pulsatile-release formulation is between 1:20 to 5:1.

An essential advantage of taking a preparation with pulsatile release kinetics is the optimal dosage of the specific nootropic agent, which can be adjusted by the user himself thanks to the precisely definable time of action. The fine dosage remains the responsibility of the individual user as it depends on his physical condition and tolerance (especially in the morning hours) on the one hand and on the time of the desired effect on the other hand. In the case of nootropic dosage, a person may not have to get up early every morning and, depending on the time of day, a certain sleep phase has occurred, which can be taken into account with the dose. In any case, the invention should make it possible to considerably facilitate the morning wake-up ritual and possibly replace a conventional alarm clock completely or reduce it to the function of a control authority. This will enable the person concerned to use the preparation in a daily manner.

In order to assure the strength of the active substance(s), the micropellets can be filled into hard or soft capsules, into sachets or into bottles to be reconstituted before administration. The micropellets can also be dispersed in a medium to form a suspension and filled into bottles, compressed as tablets that are blistered or bottled, or compressed as mini tablets that are filled into hard or soft capsules. For example, a dose of the nootropic agent being caffeine of 160 mg, for example, can be loaded into or coated onto up to several hundreds of micro pellets, in this case pellets with a size of 1000 μm, which in turn are coated with a special functional coating and release the caffeine in accordance with the chemical, physical and biological conditions present in the human digestive tract. Preferably, within the scope of the present invention, for the embodiment wherein the nootropic agent is caffeine, the strength of caffeine per dosage unit is between 10 mg and 1000 mg, more preferably between 40 mg and 320 mg, most preferably 80 mg or 160 mg. In an alternative preferred embodiment wherein the nootropic agent is caffeine, the strength of caffeine per dosage is preferably 60 mg or 160 mg.

In certain embodiments, a pulsatile-release formulation of the present invention further comprises a sedative substance. In contrast to vitalising substances, sedatives, tranquillants and hypnotics have a dampening effect on the function of the central nervous system. Sedatives are administered in cases of restlessness as a result of illness or mental disorders. Sedatives are also used before major diagnostic or therapeutic procedures to relieve a patient of stress while maintaining responsiveness. In addition, sedative substances can also be taken as a result of stress-related situations in everyday life. Such remedies are also effective against the unpleasant symptoms such as sweating, tachycardia, gastrointestinal cramps and similar accompanying symptoms of examination anxiety and other stress. Hypnotics are used to treat insomnia. Similar to vitalizing substances, sedatives must also be taken at a desired point in time immediately before the sedative substances unfold their effect. This can cause additional nervousness in a stress-exposed person who knows that the sedative has to be taken before the decisive event and only then does it take effect. The optimal timing of the intake in such situations creates unnecessary tension because the stressful person has to use part of his strength for optimized time management with regard to the sedative measures.

As understood herein, the copolymers comprised within the release-controlling polymeric system of the present invention, as well as compounds described herein, including nootropic agents and sedative substances, may be present as pharmaceutically acceptable salts. As defined herein, “Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18th ed., Mack Publishing Company, Easton, PA, 1990, p. 1445, the disclosure of which is hereby incorporated by reference.

Preferably, in the pulsatile release formulations of the present invention that further comprise a sedative substance, the said sedative substance's release is not controlled by the release controlling polymeric system of the present invention. In other words, preferably in the formulations of the present invention, the sedative substance is to be released substantially immediately upon taking the formulation. Thus, in a non-limiting example, distinct layers of a formulation of the present invention that additionally comprises a sedative substance, may be formulated as follows:
a) a core comprising sugar or microcrystalline cellulose,
b) a layer comprising a nootropic agent, e.g., caffeine,
c) a release-controlling polymeric system, and
d) a layer comprising a sedative substance.

According to the present invention, in certain embodiments the sedative substance is melatonin or a pharmaceutically acceptable salt thereof.

In certain embodiments of the present invention, the sedative substance is an antihistamine. Antihistamines are known to the skilled person and particular examples include diphenhydramine, dimenhydrinate and dimetindene. Thus, in certain embodiments of the present invention the sedative substance is diphenhydramine or a pharmaceutically acceptable salt thereof, dimenhydrinate or a pharmaceutically acceptable salt thereof, or dimetindene or a pharmaceutically acceptable salt thereof.

In certain embodiments of the present invention, the sedative substance is an antipsychotic. Antipsychotic drugs are known to the skilled person and their examples include quetiapine, levomepromazine and olanzapine. Thus, certain embodiments of the present invention the sedative substance is quetiapine or a pharmaceutically acceptable salt thereof, levomepromazine or a pharmaceutically acceptable salt thereof, or olanzapine or a pharmaceutically acceptable salt thereof.

In certain embodiments of the present invention, the sedative substance is trazodone or a pharmaceutically acceptable salt thereof. In certain embodiments of the present invention, the sedative substance is mirtazapine or a pharmaceutically acceptable salt thereof.

In certain embodiments of the present invention, the sedative substance is a z-drug. Z-drugs are known to the skilled person and particular examples include zolpidem, or zaleplon. Therefore, in certain embodiments of the present invention, the sedative substance is zolpidem or a pharmaceutically acceptable salt thereof, or zaleplon or a pharmaceutically acceptable salt thereof.

Further within the scope of the present invention encompassed are the embodiments, wherein the pulsatile-release formulation of the present invention, further comprises a sedative substance is a benzodiazepine. Preferably, the benzodiazepine is alprazolam or a pharmaceutically acceptable salt thereof, chlordiazepoxide or a pharmaceutically acceptable salt thereof, clonazepam or a pharmaceutically acceptable salt thereof, clorazepate or a pharmaceutically acceptable salt thereof, diazepam or a pharmaceutically acceptable salt thereof, estazolam or a pharmaceutically acceptable salt thereof, flurazepam (Dalmane) or a pharmaceutically acceptable salt thereof, lorazepam or a pharmaceutically acceptable salt thereof, midazolam or a pharmaceutically acceptable salt thereof, oxazepam or a pharmaceutically acceptable salt thereof, temazepam or a pharmaceutically acceptable salt thereof, triazolam or a pharmaceutically acceptable salt thereof, or quazepam or a pharmaceutically acceptable salt thereof.

In certain embodiments of the present invention, the sedative substance is doxylamine or a pharmaceutically acceptable salt thereof. In certain embodiments of the present invention, the sedative substance is chlorphenamine or a pharmaceutically acceptable salt thereof. In certain embodiments of the present invention, the sedative substance is niacin or a pharmaceutically acceptable salt thereof.

In certain embodiments of the present invention, the pulsatile-release formulation of the present invention, comprises the sedative substance, wherein the sedative substance comprises a phytochemical. As defined herein, the phytochemical is preferably selected from Valeriana officinali, Humulus lupulus, Lavandula officinalis, Hypericum perforatum, Petasites hybridus, Melissa officinalis, Passiflora incarnata, and Choisya ternata.

In certain embodiments of the present invention, the pulsatile-release formulation of the present invention, comprises the sedative substance, wherein the sedative substance comprises an amino acid selected from L-tryptophan, L-theanine, and glycine.

In certain embodiments of the present invention, the sedative substance comprises magnesium salt.

A pulsatile-release formulation of the present invention is useful in therapy. The pulsatile release formulation as defined herein is suitable for treatment of symptoms associated with sleeping disorders. Among others, these symptoms include morning depression and/or wake up-difficulties associated with a particular disease state. In particular, the pulsatile release formulation of the present invention is suitable for treatment of subjects that could benefit from the pharmacological adjustment of the circadian clock by triggering the Cortisol Awakening Response (CAR).

The diseases wherein symptoms like the morning depression and/or wake-up difficulties are manifested include, but are not limited to Delayed Sleep Phase Syndrome, Seasonal Affective Disorders, Narcolepsy, Sleep Deprivation, Depression (include major depression), Idiopathic Insomnia, or Sedative-induced Hangovers. Therefore, the present invention further encompasses the pulsatile-release formulation of the present invention for treatment of symptoms associated with Delayed Sleep Phase Syndrome, Seasonal Affective Disorders, Narcolepsy, Sleep Deprivation, Depression (include major depression), Idiopathic Insomnia, or Sedative-induced Hangovers, in particular for treatment of the morning depression and/or wake-up difficulties. Particularly useful are the pulsatile release formulations as defined herein, wherein the nootropic agent is caffeine.

The dose of caffeine, as defined herein, is preferably between 10 mg and 1000 mg, more preferably between 40 mg and 320 mg, most preferably 80 mg or 160 mg. The skilled physician is capable of determining the suitable dose based on subjects physical parameters and symptoms to be treated, among others. It is further noted that preferably, the pulsatile-release formulation, wherein the nootropic agent is caffeine, is so formulated that the strength of caffeine per dosage unit is between 10 mg and 1000 mg, more preferably between 40 mg and 320 mg, most preferably 80 mg or 160 mg. In an alternative preferred embodiment wherein the nootropic agent is caffeine, the strength of caffeine per dosage is preferably 60 mg or 160 mg.

Wake-up difficulties represent a common burden experienced by a majority of the general population. Up to 42% of adolescent report difficulties in getting-up in the morning, whereas the prevalence tends to decline with age. Shift and night workers display increased difficulties to wake-up compared to daytime workers and several psychiatric conditions—such as seasonal affective disorder—increase the intensity of sleepiness after awakening. Wake-up difficulties are often referred to as sleep inertia, a physiological state, characterized by subjective sleepiness, reduced cognitive performance and the urge to continue to sleep. This state persists for few minutes up to several hours.

Sleep inertia is a disabling state of reduced physical and mental drive following wakening, which typically lasts for less than 30 min but symptoms may persist for several hours in susceptible individuals. A large portion of healthy adolescents report persistent difficulties to rise in the morning and many shift and on-call night workers exhibit impaired performance and grogginess after waking, which raises important safety concerns in operational settings. Furthermore, impaired post-awakening drive and mood is highly prevalent in a broad range of neurological and psychiatric conditions.

Several biological and environmental factors influence the manifestation of sleep inertia. For example, abrupt wakening from deep sleep (also referred to as slow-wave sleep or stage N3 of non-rapid-eye-movement [NREM] sleep) is associated with more severe sleep inertia symptoms when compared to wakening from more superficial NREM sleep and rapid-eye-movement (REM) sleep. This finding may suggest that neurophysiological processes underlying sleep and particularly deep NREM sleep carry over into wakefulness and contribute to behavioural and cognitive deficits associated with sleep inertia. The duration of deep sleep and hence the odds of awakening from N3 sleep is increased after sleep restriction. Accordingly, abrupt wakening from recovery sleep after sleep restriction enhances sleep inertia symptoms.

As it is known to the skilled person, according to the European Pharmacopoeia Glossary, a pulsatile release dosage form, i.e., a pulsatile release formulation, is a modified-release dosage form showing a sequential, intermittent release of the active substance(s). It is further apparent to the skilled person that pulsatile release is a result of a formulation design and/or manufacturing method.

The subjects treated with the pulsatile release formulation of the present invention, in particular wherein the nootropic agent is caffeine, in particular upon administration at bedtime, show decreased subjective and objective signs of sleep inertia immediately upon morning awakening. A skilled person is capable of quantifying the signs of sleep inertia in a subject. The suitable approaches include the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI). Thus, the pulsatile release formulation of the present invention has upon oral administration at bedtime the ability to decrease subjective and objective signs of sleep inertia immediately upon morning awakening, indicated by improved scores in the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI).

The pulsatile release formulation of the present invention is characterized by a particular release profile, which can be reproduced and/or measured in the in vitro experiments. Such an experiment is showcased in the Example 1. As disclosed herein, the pulsatile-release formulation for use of the present invention, is characterized by an in vitro release profile wherein not more than 25% of a nootropic agent is released during the first 5 hours and not less than 75% of a nootropic agent is released until the 9th hour, as measured by the analysis of cumulative dissolved amount of the nootropic agent upon incubation in the dissolution media having a pH of 1.2 for 2 hours, having a pH of 6.5 for 1 hour, having a pH of 6.8 for 2 hours and having a pH of 7.2 for 5 hours.

As understood herein, the expression “until the n^{th} hour” is to be preferably understood as not earlier than after a time of between n-4 and n hours. Therefore, the term “until the 9^{th} hour” is to be construed as after a time of between 5 and 9 hours.

Preferably, a pulsatile-release formulation for use of the present invention, wherein the nootropic agent is caffeine, is characterized by an in vivo release profile in human subjects wherein the blood plasma concentration of caffeine reaches 5 μM not earlier than 4 hours following the administration.

In another embodiment, the present invention relates to a use of the pulsatile-release formulation of the present invention for targeted shifting of the circadian sleep-wake rhythm. Upon administration of the formulations of the present invention it becomes possible for the subject to shift his or her circadian clock forward or backwards in a targeted manner, thus having less difficulty getting up early in the morning. For example, if a subject normally sleeps between midnight and 8:00 a.m., they will have difficulties waking up immediately at 6:00 a.m. If, for example, the formulation of the present invention is taken at 10 p.m., this results in a caffeine release at 6 a.m. and the circadian clock is therefore shifted by 2 hours. This property of caffeine, combined with the corresponding delivery system, makes it possible to turn an individual typically active in the evening into an individual typically active in the morning without having to go through the arduous process of “getting up too early”. In addition, the formulation of the present invention offers people with jet lag a remedy for returning to their usual rhythm or adjusting to the new time-zone. In particular, the targeted shifting of the circadian sleep-wake rhythm is performed in the case of Jet Lag or Shift Work. As disclosed herein, according to the use of the present invention, the suitable formulations wherein the nootropic agent is caffeine, include the dose of caffeine is between 10 mg and 1000 mg, more preferably between 40 mg and 320 mg, most preferably 80 mg or 160 mg. In an alternative preferred embodiment wherein the nootropic agent is caffeine, the strength of caffeine per dosage is preferably 60 mg or 160 mg.

The present invention further relates to a device for the application of this preparation, which enables the input of the desired time of pharmacological vitalization or sedation in order to display the product required for this purpose on suitable hardware, such as smartphones, tablets, phablets or smartwatches, irrespective of location, according to the time of administration and the time of action. In addition, the device shall indicate the time of administration, depending on the desired time of action of such a vitalizing or sedating product, and shall display and manage all data related to pharmacological vitalization or sedation or may even trigger the time-controlled pulsatile release.

The invention is illustrated by the following examples, which however are not to be construed as limiting.

### EXAMPLES

### Materials

The following chemicals, excipients and equipment were used for the preparation of the pulsatile-release formulations of the present invention. Herein, the invention is illustrated for an embodiment wherein the nootropic agent is caffeine. A representative design of a caffeine pulsatile release pellet, according to the present invention, is illustrated in FIG. 1B.

TABLE 1
List of chemicals and excipients used for the preparation
of immediate release caffeine formulations.

| Active Substances and Excipients | Supplier |
|---|---|
| Caffeine | Siegfried |
| Povidone K30 (Kollidon ® 30) | BASF |
| Microcrystalline cellulose spheres (Cellets ®) | IPC Process Center |
| | GmbH |
| Sugar spheres (Suglets ®) | Colorcon |
| Microcrystalline cellulose (VIVAPUR ®) | JRS Pharma |
| Polyethylene glycol (CARBOWAX ™) | DOW |
| Magnesium stearate (Parteck ® LUB MST) | Merck |
| Crospovidone (Kollidon ® CL) | BASF |
| Colloidal silicon dioxide (Aerosil ®) | Evonik |
| Hydroxypropyl cellulose (Klucel ™) | Ashland |
| Citric acid anhydrous | Merck |
| Xanthan gum (Vanzan ®NF) | Vanderbilt Minerals |
| Glucose Monohydrate | Merck |

TABLE 2
List of polymers, copolymers and other excipients used
for the pulsatile-release caffeine formulations.

| Polymers, Copolymers and Excipients | Supplier |
|---|---|
| Methacrylic acid-methyl methacrylate copolymer (1:2) | Evonik |
| (Eudragit ® S) | |
| Methacrylic acid-methyl methacrylate copolymer (1:1) | Evonik |
| (Eudragit ® L) | |
| Poly (ethyl acrylate-co-methyl methacrylate-co- | Evonik |
| trimethylammonioethyl methacrylate chloride) (1:2:0.1). | |
| (Eudragit ® RS) | |
| Methyl acrylate, methyl methacrylate and methacrylic acid | Evonik |
| (7:3:1). (EUDRAGUARD ® biotic) | |
| Poly(methyl acrylate-co-methyl methacrylate-co-methacrylic | Evonik |
| acid 7:3:1) (Eudragit ® FS 30 D) | |
| Polyvinyl acetate (Kollicoat ® SR 30D) | BASF |
| Ethyl cellulose aqueous dispersion (Aquacoat ® ECD) | DuPont |
| Ethyl cellulose (Aqualon ®) | Ashland |
| Hydroxypropyl methylcellulose acetate succinate aqueous | Shin Etsu |
| dispersion (Aqoat ®) | |
| Triethyl citrate | Merck |
| Talc (Parteck ® LUB Talc) | Merck |
| Polysorbate 80 (Tween ® 80) | Merck |
| 2-propanol | Merck |
| Purified water | — |

TABLE 3
Equipment for used the production process.

| Equipment | Supplier |
|---|---|
| IKA T25 Digital Ultra-Turrax | IKA |
| IKA RW20 Digital Mechanical Overhead Stirrer | IKA |
| IKA RCT Basic Magnetic Stirrer | IKA |
| Glatt CML 10 Container Blender | Glatt |
| Glatt GS 60 Rotor Sieve | Glatt |
| Glatt TMG Vertical Granulator | Glatt |
| Glatt Mini Fluid Bed | Glatt |
| Glatt GC 1 Pan Coater | Glatt |
| Korsch XP 1 Tablet Press | Korsch |
| Erweka GTB Powder and Granulate Flow Tester | Erweka |
| Erweka SVM 122 Tapped Density Tester | Erweka |
| Erweka TAR 120 Friability and Abrasion Tester | Erweka |
| Erweka TBH 125TD Hardness Tester | Erweka |
| Retsch AS 200 Basic Sieve Shaker | Retsch |

### Preparative Example 1. Immediate Release Caffeine Formulation

Immediate release formulation was manufactured according to the protocol below and as in Tables 4 and 5.

TABLE 4
Composition of the immediate-release caffeine formulation.

| | | |
|---|---|---|
| Composition | Function | Quantity(mg/Capsule) |

Starter core

| | | |
|---|---|---|
| Microcrystalline | Starter core | 64.00 |
| cellulose spheres | | |
| (500-700 μm) | | |

Caffeine Layer

| | | |
|---|---|---|
| Caffeine | Active | 160.00 |
| | substance | |
| Povidone | Binder | 29.45 |
| Purified water | Solvent | qs |
| Total Weight | | 253.45 |

### Manufacturing of Coating Dispersion

1. Povidone is weighed and dissolved in purified water using an overhead stirrer.
2. Caffeine is added to solution of step 1 under homogenization.
3. Homogenisation of step 2 was continued for 60 minutes.
4. This suspension is further sprayed onto pellets in fluid bed processor.
5. After completion of spraying pellets are dried in fluid bed processor till the LOD was less than 2% w/w.

### Coating Process Parameters

TABLE 5
Coating process parameters of Example 1.

| Wurster size | 3″ | |
|---|---|---|
| Inlet air temperature | 60-70° | C. |
| Inlet air flow | 20-25 | m³/h |
| Product temperature | 40-45° | C. |
| Spray rate | 4 | g/min. |
| Spray atomisation pressure | 1.0-1.2 | bar |

### Preparative Examples 2 to 7-Pulsatile Release Caffeine Formulations

Pulsatile release formulations were manufactured according to the protocol below and as in Tables 6 and 7.

TABLE 6
Composition of the pulsatile-release caffeine formulation.

| | | Quantity (mg)/Capsule | | | | | |
|---|---|---|---|---|---|---|---|
| Composition | Function | Example-2 | Example-3 | Example-4 | Example-5 | Example-6 | Example-7 |
| Immediate | Core | 253.45 | 253.45 | 253.45 | 253.45 | 253.45 | 253.45 |
| release | | | | | | | |
| caffeine | | | | | | | |
| pellets | | | | | | | |
| Eudragit ® S | Release | 40.68 | 25.88 | 39.09 | 38.78 | 38.42 | 23.70 |
| | controlling | | | | | | |
| | agent | | | | | | |
| Eudragit ® L | Release | 10.17 | 6.47 | 9.77 | 9.69 | 9.61 | 5.90 |
| | controlling | | | | | | |
| | agent | | | | | | |
| Eudragit ® RS | Release | 5.65 | 4.66 | 7.63 | 8.02 | 8.48 | 7.40 |
| | controlling | | | | | | |
| | agent | | | | | | |
| Triethyl citrate | Plasticizer | 6.2 | 3.7 | 6.2 | 6.2 | 6.2 | 3.7 |
| Talc | Anti- | 3.1 | 1.85 | 3.1 | 3.1 | 3.1 | 1.85 |
| | tacking | | | | | | |
| | agent | | | | | | |
| 2-propanol | Solvent | qs | qs | qs | qs | qs | qs |
| Purified water | Solvent | qs | qs | qs | qs | qs | qs |

| | | | | | | |
|---|---|---|---|---|---|---|
| Total Weight | 319.2 | 296.0 | 319.2 | 319.2 | 319.2 | 296.0 |
| The Ratio of | 4/1/0.55 | 4/1/0.72 | 4/1/0.78 | 4/1/0.83 | 4/1/0.89 | 4/1/1.25 |
| Eudragit ® S/ | | | | | | |
| Eudragit ® L/ | | | | | | |
| Eudragit ® RS | | | | | | |

### Manufacturing of Coating Dispersion

1. 2-propanol and purified water are weighed and mixed using an overhead stirrer.
2. Eudragit® RS is added slowly into 50% of the diluent mixture in Step 1 and stirred until the polymer is completely dissolved.
3. Eudragit® S is added to solution of step 2 and stirred until the polymer is completely dissolved.
4. Eudragit® L is added to solution of step 3 and stirred until the polymer is completely dissolved.
5. Talc is added into 50% of the diluent mixture in Step 1 and stirred for 10 minutes.
6. Triethyl citrate is added to suspension of step 5 and stirred for 20 minutes.
7. The excipient suspension is poured slowly into the Eudragit® solution while stirring with an overhead stirrer.
8. This suspension is further sprayed onto pellets in fluid bed processor.
9. After completion of spraying, the pellets are dried in fluid bed processor for 30 minutes at 40° C. before filling process.

TABLE 7
Coating process parameters of Example 2-7.

| Wurster size | 3″ | |
|---|---|---|
| Inlet air temperature | 30-35° | C. |
| Inlet air flow | 20-25 | m³/h |
| Product temperature | 23-25° | C. |
| Spray rate | 4 | g/min. |
| Spray atomisation pressure | 1.0-1.5 | bar |

### Preparative Examples 8-10

Pulsatile release formulations were manufactured according to the protocol below and as in Tables 8 and 9.

TABLE 8
Composition of pulsatile-release caffeine formulation.

| | | Quantity(mg)/Capsule | | |
|---|---|---|---|---|
| | | Exam- | Exam- | Exam- |
| Composition | Function | ple- 8 | ple- 9 | ple- 10 |
| Immediate release | Core | 253.45 | 253.45 | 253.45 |
| caffeine pellets | | | | |
| Eudragit ® S | Release | 62.4 | 27.75 | 18.50 |
| | controlling agent | | | |
| Eudragit ® L | Release | 15.6 | 9.25 | 18.50 |
| | controlling agent | | | |
| Triethyl citrate | Plasticizer | 3.9 | 3.7 | 3.7 |
| Talc | Anti-tacking agent | 7.8 | 1.85 | 1.85 |
| 2-propanol | Solvent | qs | qs | qs |
| Purified water | Solvent | qs | qs | qs |
| Total Weight | | 343.1 | 296.0 | 296.0 |

| | | | |
|---|---|---|---|
| The Ratio of Eudragit ® S/Eudragit ® L | 4/1 | 3/1 | 1/1 |

### Manufacturing of Coating Dispersion

1. 2-propanol and purified water are weighed and mixed using an overhead stirrer.
2. Eudragit® S is added slowly into 50% of the diluent mixture in Step 1 and stirred until the polymer is completely dissolved.
3. Eudragit® L is added to solution of step 2 and stirred until the polymer is completely dissolved.
4. Talc is added into 50% of the diluent mixture in Step 1 and stirred for 10 minutes.
5. Triethyl citrate is added to suspension of step 4 and stirred for 20 minutes.
6. The excipient suspension is poured slowly into the Eudragit® solution while stirring with an overhead stirrer.
7. This suspension is further sprayed onto pellets in fluid bed processor.
8. After completion of spraying, the pellets are dried in fluid bed processor for 30 minutes at 40° C. before filling process.

TABLE 9
Coating process parameters of Example 8-10.

| Wurster size | 3″ | |
|---|---|---|
| Inlet air temperature | 30-35° | C. |
| Inlet air flow | 20-25 | m³/h |
| Product temperature | 23-25° | C. |
| Spray rate | 4-10 | g/min. |
| Spray atomisation pressure | 1.0-1.5 | bar |

### Preparative Example 11—Immediate Release Formulation

Immediate release formulation was manufactured according to the protocol below and as in Table 10.

TABLE 10
Composition of the immediate-release caffeine formulation.

| Composition | Function | Quantity(mg)/Capsule |
|---|---|---|
| Starter core | | |
| Microcrystalline | Starter | 64.00 |
| cellulose spheres | core | |
| (500-700 μm) | | |
| Caffeine Layer | | |
| Caffeine | Active | 160.00 |
| | substance | |
| Povidone | Binder | 32.00 |
| Purified water | Solvent | qs |
| Total Weight | | 256.00 |

The coating dispersion was prepared according to the Example 1, The coating process parameters described in Example 1 were used.

### Preparative Example 12-Pulsatile Release Formulation

Pulsatile release formulation was manufactured according to the protocol below and as in Tables 11 and 12.

TABLE 11
Composition of pulsatile-release caffeine formulation.

| Composition | Function | Quantity(mg)/Capsule |
|---|---|---|
| Immediate release | Core | 256.00 |
| caffeine pellets | | |
| Eudraguard ® biotic | Release controlling agent | 30.00 |
| Triethyl citrate | Plasticizer | 1.03 |
| Talc | Anti-tacking agent | 3.00 |
| Polysorbate 80 | Emulsifier | 0.80 |
| Purified water | Solvent | qs |
| Total Weight | | 290.83 |

### Manufacturing of Coating Dispersion

1. Purified water is weighed.
2. Polysorbate 80 is added slowly into 50% of the purified water in Step 1 and stirred until polysorbate 80 is completely dissolved.
3. The solution in Step 2 is poured slowly into the Eudraguard® biotic solution while stirring with an overhead stirrer.
4. Talc is added into 50% of the diluent mixture in Step 1 and stirred for 10 minutes.
5. Triethyl citrate is added to suspension of step 4 and stirred for 20 minutes.
6. The excipient suspension is poured slowly into the Eudraguard® biotic dispersion while stirring with an overhead stirrer and stir for 10 minutes.
7. This dispersion is further sprayed onto pellets in fluid bed processor.
8. After completion of spraying, the pellets are dried in fluid bed processor for 30 minutes at 40° C. before filling process.

TABLE 12
Coating process parameters of Example 12.

| Wurster size | 3″ | |
|---|---|---|
| Inlet air temperature | 35-45° | C. |
| Inlet air flow | 20-25 | m³/h |
| Product temperature | 25-28° | C. |
| Spray rate | 4-10 | g/min. |
| Spray atomisation pressure | 1.0-1.5 | bar |

### Preparative Example 13—Tablet Formulation

Tablet formulation was prepared according to the protocol below and as in Table 13.

TABLE 13
Composition of pulsatile-release caffeine tablet formulation.

| | | Quantity(mg)/ |
|---|---|---|
| Composition | Function | Tablet |
| Pulsatile release caffeine pellets | Active composition | 253.45 |
| Microcrystalline cellulose | Filler | 190.55 |
| Povidone | Binder | 12.00 |
| Crospovidone | Disintegrant | 14.00 |
| Polyethylene glycol | Lubricant | 5.00 |
| Magnesium stearate | Lubricant | 5.00 |
| Core Tablet Weight | | 480.00 |
| Film coating material | Coating agent | 10.00 |
| Film Coated Tablet Weight | | 490.00 |

### Manufacturing of Tablet Formulation

1. Pulsatile release caffeine pellets are manufactured described in example 7.
2. Pulsatile release caffeine pellets, microcrystalline cellulose, povidone and crospovidone are mixed for 20 minutes in blender.
3. Polyethylene glycol and magnesium stearate are sifted and added to the blend of step 2 and mixed for 5 minutes in blender.
4. The blend was compressed on a rotary compression machine using 15×8 mm oval standard concave punches.
5. The core tablets are coated with film coating materials in the coating machine.

### Preparative Example 14-Minitablet Formulation

Minitablet formulation was prepared according to the protocol below and as in Table 14.

TABLE 14
Composition of pulsatile-release caffeine minitablet formulation

| | | Quantity (mg)/ |
|---|---|---|
| Composition | Function | Minitablet |
| Pulsatile release caffeine pellets | Active | 5.28 |
| | composition | |
| Microcrystalline cellulose | Filler | 0.98 |
| Povidone | Binder | 0.80 |
| Crospovidone | Disintegrant | 0.70 |
| Colloidal silicon dioxide | Glidant | 0.08 |
| Polyethylene glycol | Lubricant | 0.08 |
| Magnesium stearate | Lubricant | 0.08 |
| Core Tablet Weight | | 8.00 |
| Film coating material | Coating agent | 0.20 |
| Film Coated Tablet Weight | | 8.20 |

### Manufacturing of Minitablet Formulation

1. Pulsatile release caffeine pellets are manufactured described in example 7.
2. Pulsatile release caffeine pellets, microcrystalline cellulose, povidone and crospovidone are mixed for 20 minutes in blender.
3. Colloidal silicon dioxide is sifted and added to the blend of step 2 and mixed for 5 minutes in blender.
4. Polyethylene glycol and magnesium stearate are sifted and added to the blend of step 3 and mixed for 5 minutes in blender.
5. The blend was compressed on a rotary compression machine using 2.3 mm circular standard concave punches.
6. The core tablets are coated with film coating materials in the coating machine.

393.6 mg of the thus obtained minitablets each, corresponding to 160 mg caffeine may fill into the hard gelatine capsules, or sachets or a bottle.

### Preparative Example 15—Oral Suspension Formulation

Oral suspension formulation was prepared according to the protocol below and as indicated in Table 15 and 16.

TABLE 15
Composition of pulsatile-release caffeine
oral suspension formulation

| | | Quantity |
|---|---|---|
| Composition | Function | mg/5 mL |
| Pulsatile release caffeine | Active composition | 253.45 |
| pellets | | |
| Hydroxypropyl cellulose | Suspending and viscosity- | 3.50 |
| | increasing agent. | |
| Citric acid anhydrous | pH adjusting agent | 10.00 |
| Xanthan gum | Suspending agent | 3.70 |
| Glucose Monohydrate | Sweetening agent | 2104.35 |
| Purified water | Solvent | qs to 5 mL |

### Manufacturing of Oral Suspension Formulation

1. Pulsatile release caffeine pellets are manufactured described in example 1 or 2.
2. Glucose monohydrate is weighed and dissolved in purified water using an overhead stirrer.
3. Citric acid anhydrous is added into solution of step 2 and stir until completely dissolved.
4. Hydroxypropyl cellulose is added into solution of step 3 and stir until completely dissolved.
5. Xanthan gum is added into solution of step 4 and stir until completely dissolved.
6. Pulsatile release caffeine pellets is added into solution of step 5 and stir for 10 minutes.
7. After the final mixing, oral suspension is filled into a bottle.

TABLE 16
In vitro release test parameters

| Media | pH 1.2 (2 h) - pH 6.5 (1 h) - pH 6.8 (2 h) - pH | |
|---|---|---|
| | 7.2 (5 h) | |
| Time Points | For pH 1.2: 2^{nd} hour | |
| | For pH 6.5: 1^{st} hour | |
| | For pH 6.8: 2^{nd} hour | |
| | For pH 7.2: 1^{st} hour, 2^{nd} hour, | |
| | 4^{th} hour and 5^{th} hour | |
| Surfactant | None | |
| Stirring Speed | 100 | rpm |
| Apparatus | Basket | |
| Media Volume | 900 ± 1 | mL |
| Temperature | 37 ± 0.5° | C. |

### Reference Preparative Example 1

Pulsatile release formulations were manufactured according to the protocol below and as in Table R1 and R2.

TABLE R1
Composition of the pulsatile-release caffeine formulation.

| | | Quantity(mg)/ |
|---|---|---|
| Composition | Function | Capsule |
| Immediate release | Core | 253.45 |
| caffeine pellets | | |
| Eudragit ® S | Release controlling agent | 23.7 |
| Eudragit ® L | Release controlling agent | 5.9 |
| Eudragit ® RS | Release controlling agent | 19.65 |
| Triethyl citrate | Plasticizer | 4.23 |
| Talc | Anti-tacking agent | 2.17 |
| 2-propanol | Solvent | qs |
| Purified water | Solvent | qs |
| Total Weight | | 309 |

| | |
|---|---|
| The Ratio of Eudragit ® S/Eudragit ® L/Eudragit ® RS | 4/1/3.33 |

### Manufacturing of Coating Dispersion

1. 2-propanol and purified water are weighed and mixed using an overhead stirrer.
2. Eudragit® RS is added slowly into 50% of the diluent mixture in Step 1 and stirred until the polymer is completely dissolved.
3. Eudragit® S is added to solution of step 2 and stirred until the polymer is completely dissolved.
4. Eudragit® L is added to solution of step 3 and stirred until the polymer is completely dissolved.
5. Talc is added into 50% of the diluent mixture in Step 1 and stirred for 10 minutes.
6. Triethyl citrate is added to suspension of step 5 and stirred for 20 minutes.
7. The excipient suspension is poured slowly into the Eudragit® solution while stirring with an overhead stirrer.
8. This suspension is further sprayed onto pellets in fluid bed processor.
9. After completion of spraying, the pellets are dried in fluid bed processor for 30 minutes at 40° C. before filling process.

TABLE R2
Coating process parameters of reference preparative example 1.

| Wurster size | 3″ | |
|---|---|---|
| Inlet air temperature | 30-35° | C. |
| Inlet air flow | 20-25 | m³/h |
| Product temperature | 23-25° | C. |
| Spray rate | 4 | g/min. |
| Spray atomisation pressure | 1.0-1.5 | bar |

### Reference Preparative Example 2

Pulsatile release formulation was manufactured according to the protocol below and as in Table R3 and R4.

TABLE R3
Composition of the pulsatile-release caffeine formulation.

| Composition | Function | Quantity(mg)/Capsule |
|---|---|---|
| Immediate release | Core | 256.00 |
| caffeine pellets | | |
| Eudragit ® FS 30 D | Release controlling agent | 30.00 |
| Triethyl citrate | Plasticizer | 1.50 |
| Talc | Anti-tacking agent | 15.00 |
| Purified water | Solvent | qs |
| Total Weight | | 302.50 |

### Manufacturing of Coating Dispersion

1. Purified water is weighed.
2. Talc and triethyl citrate are added slowly into the purified water in Step 1 and homogenized for 10 minutes.
3. The solution in Step 2 is poured slowly into the Eudragit® FS 30 D dispersion while stirring with an overhead stirrer and stir for 10 minutes.
4. The dispersion in Step 3 is passed through a 0.5 mm sieve.
5. This dispersion is further sprayed onto pellets in fluid bed processor.
6. After completion of spraying, the pellets are dried in fluid bed processor for 30 minutes at 40° C. before filling process.

TABLE R4
Coating process parameters of reference preparative example 2.

| Wurster size | 3″ | |
|---|---|---|
| Inlet air temperature | 35-45° | C. |
| Inlet air flow | 20-25 | m³/h |
| Product temperature | 25-28° | C. |
| Spray rate | 4-10 | g/min. |
| Spray atomisation pressure | 1.0-1.5 | bar |

### Example 1. In Vitro Release Measurements of Formulations According to Preparative Experiments 2 to 8 and 12

The test parameters were simulated considering the gastric and intestinal conditions and the formulation was developed using a pH and time-dependent approach. For simulating conditions of the GI tract, dissolution studies were carried out in media with pH 1.2 (HCl 0.1N), pH 6.5, pH 6.8 and pH 7.2 (phosphate buffer) and samples were tested separately at each medium. Dissolution time was 2 h for medium with pH 1.2, 1 h for medium with pH 6.5, 2 h for medium with pH 6.8 and 5 h for the pH 7.2.

In-vitro test parameters are given below and for formulation according to the Preparative example 7 are illustrated in FIG. 1C.

| Media | pH 1.2 (2 h) - pH 6.5 (1 h) - pH 6.8 (2 h) - pH | |
|---|---|---|
| | 7.2 (5 h) | |
| Time Points | For pH 1.2: 2^{nd} hour | |
| | For pH 6.5: 1^{st} hour | |
| | For pH 6.8: 2^{nd} hour | |
| | For pH 7.2: 1^{st} hour, 2^{nd} hour, | |
| | 4^{th} hour and 5^{th} hour | |
| Surfactant | None | |
| Stirring Speed | 100 | rpm |
| Apparatus | Basket | |
| Media Volume | 900 ± 1 | mL |
| Temperature | 37 ± 0.5° | C. |

TABLE 17
In vitro release of Example 2-8 and Example 12 (Average ± Standard Deviation).

| Preparative | | | | | | | |
|---|---|---|---|---|---|---|---|
| Example | pH 1.2 | pH 6.5 | pH 6.8 | pH 7.2 | pH 7.2 | pH 7.2 | pH 7.2 |
| Number | (2 hours) | (1 hour) | (2 hours) | (1 hour) | (2 hours) | (4 hours) | (5 hours) |
| Example 2 | 2 ± 0.9 | 5 ± 1.7 | 12 ± 1.6 | 19 ± 1.4 | 28 ± 1.6 | 60 ± 2.0 | 74 ± 1.7 |
| Example 3 | 9 ± 0.2 | 19 ± 0.4 | 41 ± 0.4 | 79 ± 1.0 | 89 ± 1.2 | 91 ± 0.7 | 91 ± 0.8 |
| Example 4 | 1 ± 0.0 | 7 ± 0.2 | 20 ± 0.9 | 38 ± 1.4 | 67 ± 2.0 | 95 ± 0.4 | 96 ± 0.3 |
| Example 5 | 2 ± 0.2 | 7 ± 0.3 | 17 ± 0.6 | 29 ± 1.1 | 53 ± 1.3 | 95 ± 1.4 | 98 ± 1.5 |
| Example 6 | 1 ± 0.2 | 5 ± 0.8 | 16 ± 1.2 | 31 ± 1.5 | 74 ± 2.7 | 99 ± 1.0 | 99 ± 0.8 |
| Example 7 | 3 ± 0.2 | 5 ± 0.6 | 10 ± 0.8 | 16 ± 1.0 | 49 ± 0.9 | 101.3 ± 1.3 | 101.7 ± 1.7 |
| Example 8 | 3 ± 0.2 | 10 ± 0.2 | 35 ± 0.3 | 83 ± 0.7 | 90 ± 0.9 | 91 ± 0.8 | 91 ± 0.8 |
| Example 12 | 1 ± 0.2 | 2 ± 0.5 | 3 ± 0.7 | 93 ± 1.5 | 94 ± 1.5 | 94 ± 1.0 | 94 ± 1.6 |

### Example 2. Clinical Study

Herein we developed a pulsatile-release caffeine delivery system that releases the stimulant shortly before waking, to target immediate improvements of impaired subjective state, vigilance and performance due to sleep inertia. We hypothesized that oral bedtime intake of this innovative caffeine pulsatile-release formulation, aimed to start releasing an efficacious dose of caffeine with a delay of approximately 7 hours, facilitates the sleep-to-wake transition by preventing sleep inertia (rather than treating). We tested this hypothesis in two randomized, double-blind, cross-over, placebo-controlled studies. First, we examined the in vivo release properties of the engineered caffeine pulsatile-release formulation throughout a nocturnal sleep episode. Then, we comprehensively investigated its effects on behavioural, emotional, cognitive and physiological symptoms of sleep inertia in sleep-restricted healthy young men. We expected that the delayed, pulsatile release of caffeine prior to wakening improves vigilance and mood immediately after wake-up, elevates CAR and reduces sleep propensity in a scheduled morning nap opportunity 1 hour after wakening.

To test this notion, we here perform two separate, randomized, double-blinded, cross-over, placebo-controlled studies in healthy volunteers. In a first study, the in vivo release properties of the engineered caffeine formula are examined in 10 healthy subjects by means of continuous blood sampling. In a second study, we investigate the effects of the validated formula on post-awakening vigilance (Psychomotor Vigilance Task; PVT), working memory (N-Back task), sustained attention (D2 task), mood (Positive and Negative Affective Schedule), the cortisol awakening response (CAR) and the tendency to continue sleep (sleep latency task) in 22 healthy volunteers. Since pharmacological challenges in healthy volunteers often suffer from neuropsychological ceiling/floor effects, subjects are additionally sleep restricted (sleep from 3:00-7:00 instead of 23:00-7:00) to experimentally increase sleep pressure and accordingly sleep inertia upon awakening. Additionally, blood is continuously sampled to monitor individual caffeine release profiles. Night-time sleep is monitored by means of sleep electroencephalography (EEG).

Based on above outline, we hypothesize, that our formula mitigates the build-up of sleep inertia, indicated by enhanced post-awakening vigilance and mood immediately after awakening, an elevated CAR and prolonged sleep latencies. With that, we here propose an innovative tool, which may help to facilitate sleep-wake transition in healthy individuals, but potentially also in a clinical population suffering from disabling sleep inertia.

### Methodology

### Participants and Permissions

A total of 32 healthy young men (mean age: 25.6±3.7 years) participated in the two studies (in vivo validation study: n=10; pharmacodynamic study: n=22), whereof 5 subjects participated in both studies. Following criteria were required for inclusion: (i.) male sex in order to avoid the potential impact of menstrual cycle on sleep physiology or HPA axis activity, (ii.) age within the range of 18 to 34 years, (iii.) a body-mass-index below 25, (iv.) an Epworth Sleepiness Score (ESS) below 10, (v.) habitual sleep onset latency below 20 minutes, (vi.) regular sleep-wake rhythm with bedtime between 11 pm and 1 am, (vii.) absence of any somatic or psychiatric disorders, (viii.) no acute or chronic medication intake, (ix.) non-smoking, (x.) no history of drug abuse (lifetime use>5 occasions, with exception of occasional cannabis use), (xi.) caffeine consumption of less than 4 units per day (coffee, tea, chocolate, cola, energy drinks). Participants had to abstain from illegal drugs and caffeine for two weeks prior to the experimental night. No alcohol was allowed 24 h before the experimental night. Participants were instructed to keep an individual regular sleep-wake rhythm (23:00-7:00 or 22:00-6:00 depending on the volunteers' habitual bedtime) during the entire study, starting two weeks prior to the experimental night.

To ensure adherence to the regular sleep-wake pattern, participants were instructed to wear a rest-activity monitor on the non-dominant arm and to keep a sleep-wake diary. The studies were approved by the Cantonal Ethics Committee of the Canton of Zurich (ID: 2018-00533). All participants provided written informed consent according to the declaration of Helsinki.

### Study Procedures

In a first evaluation study of the engineered caffeine formulation, the in vivo caffeine release profile was determined in 10 male individuals. After oral intake at 22:30, study participants were allowed to sleep from 23:00-7:00, while blood was continuously sampled. Samples were collected from the left antecubital vein at baseline (22:00), and 1.5, 2.5, 3.5, 4.5, 5.5, 6.5, 7.5, 8.5, 9.5, 10.5, 13.5 and 17.5 hours after drug administration. During the sleep episode in the soundproof and climatized bedrooms of the sleep laboratory, the venous catheter was connected to a blood-collection setup in an adjacent room (Heidelberger plastic tube extensions through the wall). Thus, blood samples (4 ml, BD Vacutainer EDTA) were collected without disturbing the sleeping study participants. The intravenous line was kept patent with a slow drip (10 ml/h) of heparinized saline (1000 IU heparin in 0.9 g NaCl/dL; HEPARIN Bichsel; Bichsel AG, 3800 Unterseen, Switzerland). Blood samples were immediately centrifuged for 10 min at 2000 RCF and plasma samples were immediately stored on ice until final storage at −80° C.

### Analysis of Caffeine Levels

Caffeine and caffeine-13C3 were purchased from Sigma-Aldrich (St. Louis, USA). All chemicals used were of the highest purification grade available. Briefly, 200 μl of plasma, 50 μl of the internal standard (IS) (40 μM caffeine-13C3) and 50 μl of methanol (MeOH) were added to an Eppendorf tube. For protein precipitation, 400 μl of acetonitrile (ACN) were slowly added. Samples were shaken for 10 min and centrifuged at 10′000 rpm for 5 min. 350 μl of the supernatant was transferred into an auto-sampler vial and evaporated to dryness under a gentle stream of nitrogen. For reconstitution, 250 μl of an eluent-mixture (95:5, v/v) was added. Quality control (QC) samples and calibrators (cal) were prepared with the same sample preparation, replacing the 50 μl of MeOH with the Cal or QC solutions. The plasma samples were analyzed on an ultra-high performance liquid chromatography (UHPLC) system (Thermo Fisher, San Jose, CA) coupled to a linear ion trap quadrupole mass spectrometer 5500 (Sciex, Darmstadt, Germany). The mobile phases of the UHPLC consisted of water (eluent A) and a mixture (70:30 v/v) of MeOH and ACN (eluent B), both containing 0.1% of formic acid (v/v) and 5 μl of sample were injected. Using a Kinetex Biphenyl column (50×2.1 mm, 1.7 μm) (Phenomenex, Aschaffenburg, Germany), The flow rate was set to 0.45 mL/min with the following gradient: start conditions 95% of eluent A, decreasing to 80% in 3 min followed by a quick decrease to 2% within 0.5 min. These conditions were hold for 1 min and switched to the starting conditions for re-equilibration for 1 min. The mass spectrometer was operated in positive electrospray ionization mode with scheduled multiple reaction monitoring. Three MRM transitions were used for both analytes. For quantification, the peak area of the analytes were further integrated and divided by the peak area of the IS. Cal samples were fitted with a least squares fit and weighted by 1/x.

### Study Drug

The caffeine pulsatile-release formulation is manufactured using the drug layering process. In this process, caffeine and the excipients are dispersed in the coating media and then sprayed onto inert microcrystalline cellulose spheres using a fluid bed through a Wurster tube with continuous inlet air that dries the liquid in the dispersion, to obtain various layers consisting of caffeine, release-controlling polymeric system and other excipients. After obtaining the micropellets, they are encapsulated into hard capsules. The release-controlling polymeric system is based on methacrylate copolymers, which controls the release of caffeine both pH-dependently and pH-independently. The release mechanism of the polymeric system is mainly driven by the swellability and permeability of the copolymers.

The in vitro dissolution profiles of different prototypes were tested by means of state-of-the-art dissolution assays, mimicking gastrointestinal conditions. Development and in vitro testing of the caffeine pulsatile-release formulation and placebos was conducted at Elixir Pharmaceutical Research and Development Corporation in Ankara, Turkey. For the in vivo study, the most suited prototype with a favourable in vitro dissolution profile was chosen. The formulation according to the Preparative Example 7 shows the best in vitro dissolution profile (see Table 17 and FIG. 1C) as a promising pulsatile release formulation. Therefore, this example was used in pharmacodynamic study.

### Pharmacodynamic Study

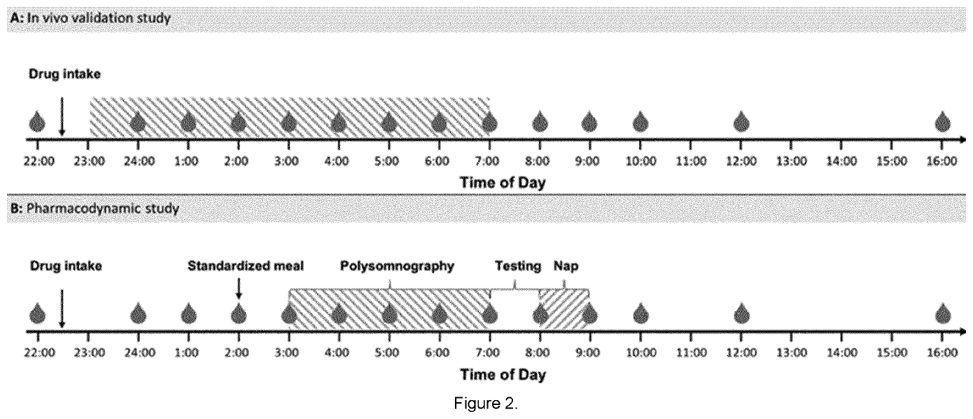

In the pharmacodynamic study, the pulsatile-release caffeine formulation (which was validated in vivo as described above) or a placebo (matched in appearance) were administered at 22:30. To exacerbate sleep inertia symptoms and avoid neuropsychological ceiling/floor effects, which are frequent in interventions studies with highly functioning healthy volunteers, all participants were sleep restricted. More specifically, they were kept awake until 3:00, then given a 4-hour sleep opportunity, and awoken at 7:00. At 2:00, all volunteers received a standardized, light meal. Blood was continuously sampled upon drug administration and the caffeine release from the formulation was monitored as described above. Upon awakening, the effects of the formulation on subjective, neuropsychological, emotional and endocrinological markers of sleep inertia were assessed. Additionally, physiological sleep tendency was investigated by determining the sleep characteristics of a 1-hour nap opportunity starting at 8:00. Both studies followed a randomized, double-blind, placebo controlled, crossover design. The details of both study designs are illustrated in FIG. 2.

### Test Battery

A comprehensive test battery including the following validated questionnaires, tasks and physiological measures to assess subjective, behavioural, cognitive, emotional and endocrinological markers of sleep inertia was administered immediately upon wakening (07:00-08:00).

Modified Sleep Inertia Questionnaire (SIQ): We modified the SIQ (Kanady and Harvey, 2015) to assess volunteers' subjective experience of the awakening process. The original version of the SIQ represents a trait inventory, in which subjects are instructed to rate the quality of their awakening process of the last week, namely on a physiological, emotional, cognitive and behavioural level. Thereby, the inventory instruction reads as follows: “On a typical morning in the past week, after you woke up, to what extent did you e.g., have problems to get out of bed” (possible ratings: 1) not at all, 2) a little, 3) somewhat, 4) often, 5) all the time). For the present study, we reformulated the inventory's instruction to gain state information of the wake-up process of the experimental morning (rather than trait information of the last week) in order to analyze the acute effects of our delayed-release formula. To this end, the instruction was rearticulated as follows: “How strong did you feel the following aspects after you woke up this morning compared to a normal morning last week: e.g., have problems to get out of bed” (possible ratings: extremely less (−3), much less (−2), a little bit less (−1), same (0), a little bit more (+1), much more (+2), extremely more (+3). Our modified version of the SIQ (renamed to Acute Sleep Inertia Questionnaire (ASIQ) was applied at 7:45 am.

Caffeine Acute Questionnaire (CAQ): To measure specific caffeine-related effects, the CAQ was applied at 7:30.

Positive and Negative Affective Schedule (PANAS): The PANAS (Watson et al., 1988) was used to assess mood 15 minutes after awakening (at 7:15 am).

Psychomotor Vigilance Test (PVT): Vigilance was assessed with a 10-min version of the PVT at 7:05 am, immediately after wakening. The median reaction time (RT) and the numbers of lapses (trials with RT>500 ms) were analyzed.

### Cortisol Awakening Response (CAR)

Saliva of each subject was sampled at time points 7:00 (immediately after wakening), 7:15, 7:30, 7:45, and 8:00 a.m. Thereby, subjects were instructed to chew the swab for 60 s and then return it into the Salivette® tube (Sarstedt, Germany). After sampling, tubes were immediately stored on ice until final storage at −80° C. For cortisol detection, tubes were defrosted and centrifuged for 5 minutes at 5′000 rpm to yield clear saliva in the conical tube. Two subjects had to be excluded, as the amount of saliva yielded from the swabs was insufficient. Then, the swab was removed, and the yielded saliva was used for further analysis. A liquid-liquid extraction was carried out by adding 1.5 ml ethyl acetate to 265 μl of saliva sample and 50 μl IS (Cortison-D7 0.1 ng/μL). The resulting mixture was subsequently shaken for 10 min at 5 Hz. The samples were centrifuged for 5 min at 9000 rpm and then placed in a freezer (−20° C.) for approximately 60 min. The ethyl acetate layer was poured off and dried under nitrogen at 35° C. The dry residue was re-suspended using 150 μl methanol and 350 μl ammonium formate (5 mM) solution, which was used for liquid chromatography-tandem mass spectrometry (LC-MS/MS) analysis following a recently published method using 13C3-labeled cortisol as surrogate analyte for calibration (Binz, T. M., Braun, U., Baumgartner, M. R., Kraemer, T., 2016. Development of an LC-MS/MS method for the determination of endogenous cortisol in hair using 13C3-labeled cortisol as surrogate analyte. J. Chromatogr. B Anal. Technol. Biomed. Life Sci.). The method was validated according to the guidelines of the German Society of Toxicology and Forensic Chemistry (GTFCh) (Peters et al., F. T., 2009. Anhang B zur Richtlinie der GTFCh zur Qualitätssicherung bei forensisch-toxikologischen Untersuchungen-Anforderungen an die Validierung von Analysenmethoden Analysenmethoden. Toxichem. Krimtech. https://doi.org/10.1007/s00265-014-1867-8). The limit of detection for cortisol was 0.55 nmol/l and the limit of quantification was 1.1 nmol/l.

### Polysomnography

As in previous study (Dornbierer, D. A., Baur, D. M., Stucky, B., Quednow, B. B., Kraemer, T., Seifritz, E., Bosch, O. G., Landolt, H. P., 2019. Neurophysiological signature of gamma-hydroxybutyrate augmented sleep in male healthy volunteers may reflect biomimetic sleep enhancement: a randomized controlled trial. Neuropsychopharmacology. https://doi.org/10.1038/s41386-019-0382-z), nocturnal sleep from 23:00-07:00 (in vivo caffeine-release study) and from 03:00-07:00 (pharmacodynamic study) was quantified by all-night polysomnography with Rembrandt® Datalab (Version 8; Embla Systems, Planegg, Germany). In the pharmacodynamic study, sleep pressure after wakening was also assessed by determining the participants' sleep onset latency (SOL) and sleep patterns during a nap opportunity starting at 8:00 (FIG. 1A). The recording setup consisted of 10 EEG electrodes (Fp1, Fp2, F3, F4, C3, C4, P3, P4, O1, O2) according to the 10-20 system, a bipolar electrooculogram (EOG), a submental electromyogram (EMG) and an electrocardiogram (ECG). The individual EEG electrode coordinates were marked by cutting a few hairs at the electrode positions, to ensure that the electrodes were placed at the very same place in both experimental conditions.

All data were recorded with dedicated polygraphic amplifiers (Artisan®, Micromed, Mogliano Veneto, Italy). As in previous studies, the analog signals were conditioned by a high-pass filter (EEG:−3 dB at 0.15 Hz; EMG: 10 Hz; ECG: 1 Hz) and an antialiasing low-pass filter (−3 dB at 67.2 Hz), digitized and stored with a resolution of 256 Hz (sampling frequency of 256 Hz) (Dornbierer et al., Neuropsychopharmacol, 2019).

### Visual Sleep Scoring

For sleep scoring, the C3-A2 derivation was used. Sleep variables were visually scored based on 30-s epochs according to the criteria of the American Academy of Sleep Medicine. Movement- and arousal-related artifacts were visually identified and excluded from the analyses. The following sleep variables were computed: time spent in (i) wakefulness (Wake), (ii) stage 1 (N1), (iii) stage 2 (N2), (iv) stage 3 (N3), (v) stage REM, (vi) sleep onset latency (SOL=time between lights-off and first occurrence of N1) and (vii) sleep efficiency (SEFF=[TST/TIB]*100; TST=time spent in N1, N2, N3 and REM sleep; TIB=time between lights-off and lights-on).

### Statistical Analyses

Independent linear mixed-effects models, with condition (caffeine vs. placebo) as within-subject factor and subject ID as random effect were employed on R (RStudio Version 1.0.136; RStudio, Inc.) for the analysis of the 1) ASIQ, 2) CAQ, 3) PANAS, 4) PVT, 5)N-Back, 6) D2 task, 7) CAR, 8) nocturnal sleep variables and 9) morning nap variables (R-package “Ime4,” Version 1.1-15). For all applied models, normal Q-Q plots were applied, demonstrating normality of the residuals. Moreover, the assumption of homoscedasticity and linearity was verified using a Tukey-Anscombe (residuals vs. fitted) plot. Post hoc testing was carried out using the R package emmeans (Version 1.2.1). P-values of post-hoc tests (R-package “emmeans,” Version 1.2.1) were corrected for multiple comparison using Benjamini-Hochberg correction of the false discovery rate. If not noted otherwise, only significant effects and differences are reported.

### Results

### Caffeine Release Profile

The formulation in vivo validation study revealed a pulsatile-release profile of the administered formulation, whereas Cmax (maximal plasma concentration) was reached after 10.5 h. The caffeine curve followed a sustained-release profile, whereas efficacious plasmatic levels (>5 μM) were attained after 7 hours.

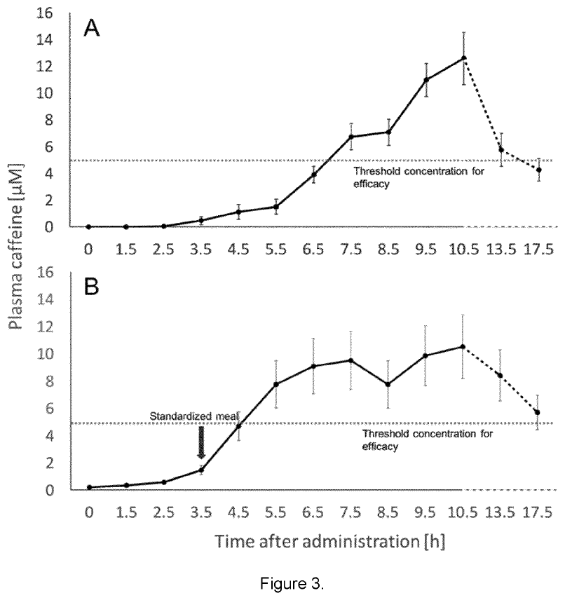

Unexpectedly, in the pharmacodynamic study, the caffeine release profile distinctly differed from that in the in vivo validation study. A sustained-release of caffeine already started after 3.5 hours and attained efficacious plasma caffeine levels 5 hours post-administration. This premature burst was most likely triggered by gastric movements due to a standardized meal served to the subjects 3.5 hours post-administration (FIG. 3). It is therefore noted that eating should be avoided after administration of the formulation of the present invention.

### Subjective, Behavioural, Cognitive, Emotional and Physiological Symptoms of Sleep Inertia.

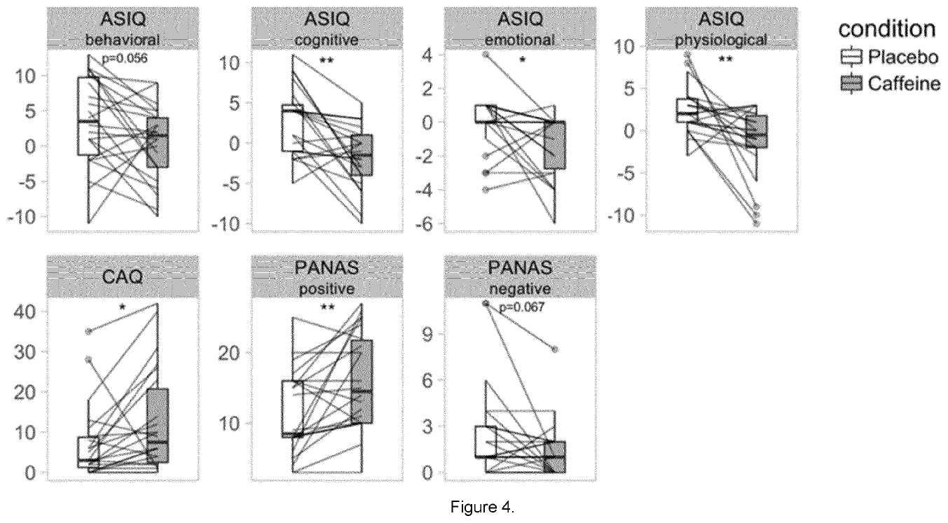

ASIQ. The statistical analyses revealed a significant condition effect (F=31.210; p<0.001; η²=0.175), such that the engineered caffeine-release formula reduced the ratings on all subscales of the ASIQ (behavioural, cognitive, emotional; physiological; FIG. 3). Remarkably, several individuals reported less problems to rise from bed on the experimental mornings compared to a normal morning in the week preceding the experiment (indicated as negative values in FIG. 4). This notion was particularly true during the caffeine condition (n=number of subjects with negative values: behavioural n=9, cognitive n=12, emotional n=9, and physiological n=10) and to a minor degree also during the placebo condition (behavioural n=5, cognitive n=7, emotional n=5, and physiological n=2).

CAQ. The statistical analyses revealed significantly increased CAQ ratings in the caffeine condition when compared to placebo (F=5.135; p=0.034; η²=0.196; FIG. 4). PANAS. The statistical analyses revealed a significant condition*item interaction (F=8.490; p=0.004; η²=0.118), such that the engineered caffeine pulsatile-release formulation increased positive ratings and reduced negative ratings when compared to placebo (FIG. 4).

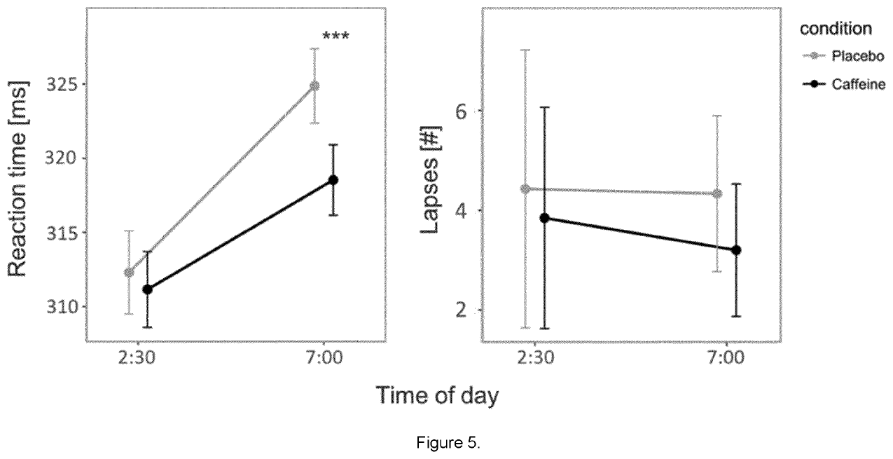

PVT. The statistical analyses of the PVT data revealed a significant condition*timepoint interaction for median reaction time (F=29.445; p<0.001), such that caffeine improved PVT median reaction time by roughly 10 ms when compared to placebo. On the other hand, the number of lapses remained unaffected (F=1.0391; p=0.359) (FIG. 5).

N-back and D2 Tasks. The statistical analyses of the n-back working memory (F=0.43; p>0.05) and the D2 sustained attention tasks performance (F=0.2892; p>0.05; η²=0.001) revealed no significant condition effects (data not shown).

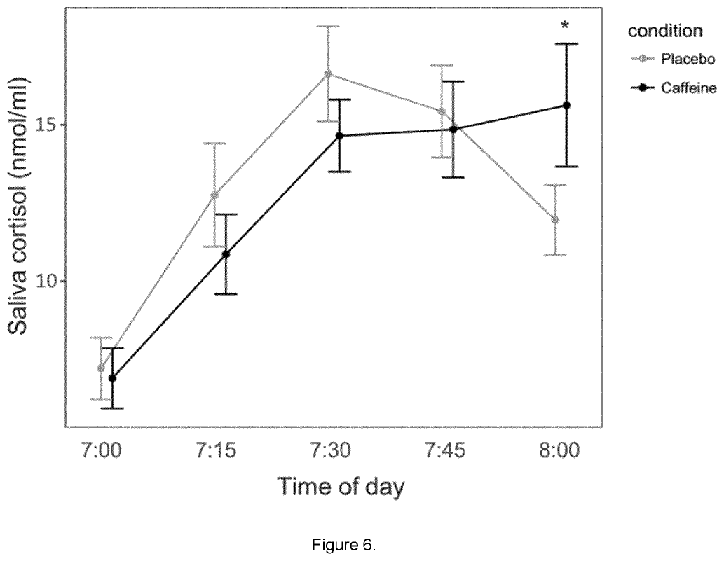

CAR. The statistical analyses of the salivary cortisol levels revealed a significant condition effect, such that cortisol levels were increased at 8:00 in the caffeine condition when compared to placebo condition (FIG. 6).

### Sleep Variables

Sleep variables are summarized in Table 18.

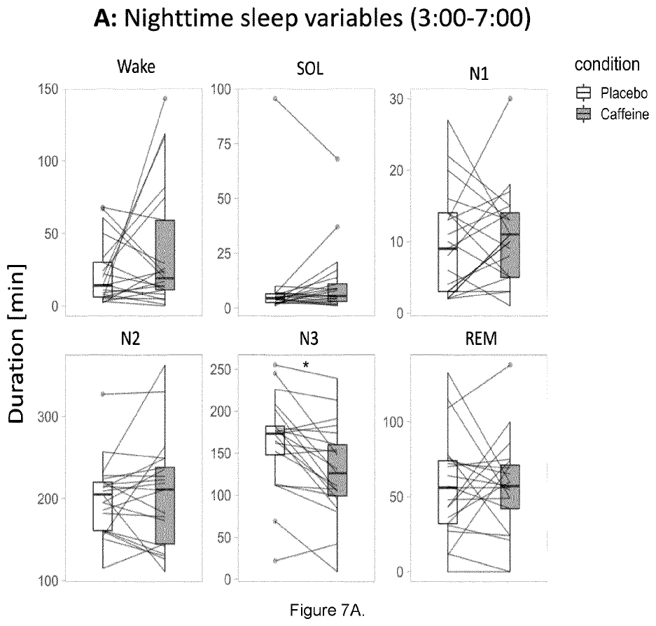

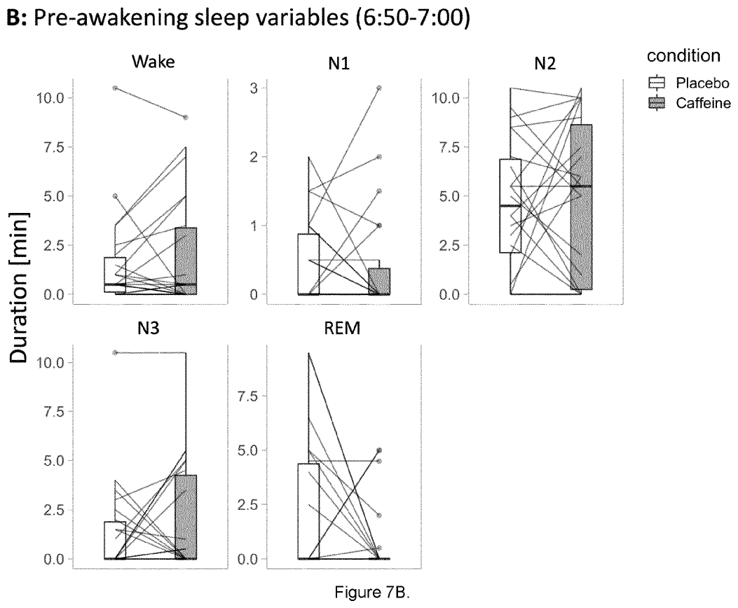

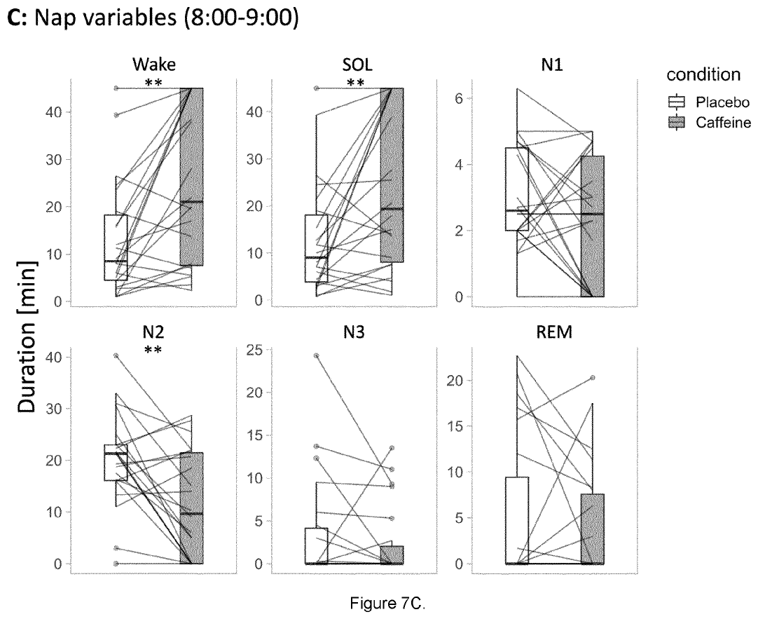

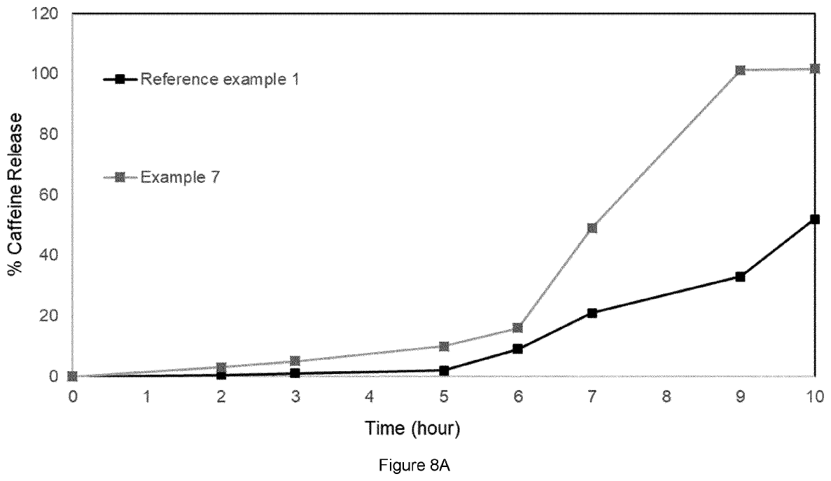

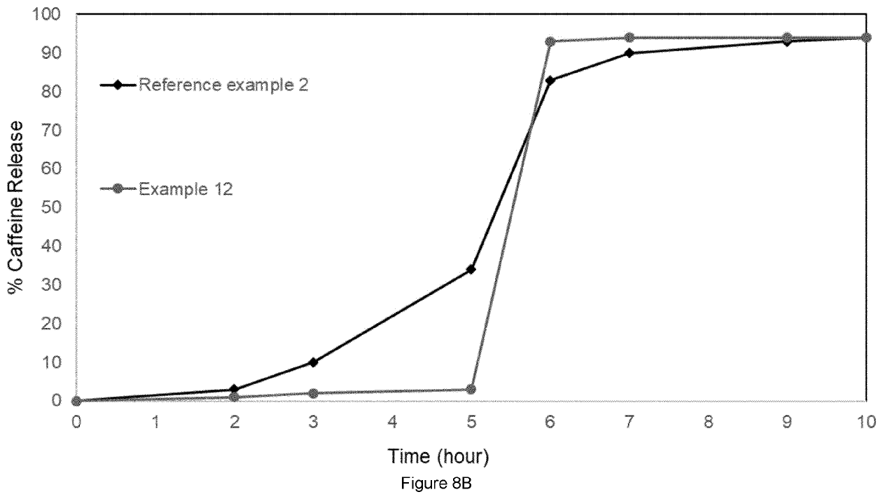

Nocturnal sleep. Given that the duration of wakefulness was experimentally prolonged prior to the initiation of nocturnal sleep, participants showed a short sleep onset latency and a high amount of deep stage N3 sleep. The statistical analyses revealed no significant main effect (F=0.558; p>0.05; η²=0.0025). Nevertheless, post hoc testing indicated that time spent in N3 sleep was shorter (p=0.004) in the caffeine condition when compared to the placebo condition (FIG. 7A).

Nap sleep. The statistical analyses of the sleep variables during the nap opportunity 1 hour after scheduled awakening revealed a significant condition effect (F=2.0875; p=0.009; η²=0.87), such that the SOL (p=0.009) and wakefulness after sleep onset (p=0.005) were prolonged, while the time spent in stage N2 was reduced (p=0.005) in the caffeine condition when compared to the placebo condition (FIG. 7B).

TABLE 18
Sleep variables.

| | Placebo | | Caffeine | | | | BH. |
|---|---|---|---|---|---|---|---|
| Variables | Mean | SD | Mean | SD | df | t-value | p.value |

Nighttime sleep

| | | | | | | | |
|---|---|---|---|---|---|---|---|
| Wake [min] | 22.333 | 21.553 | 38.048 | 43.399 | 20 | 1.511 | 0.439 |
| SOL [min] | 8.810 | 19.984 | 11.405 | 15.449 | 20 | 1.084 | 0.583 |
| N1 [min] | 9.714 | 7.343 | 10.810 | 6.501 | 20 | 0.641 | 0.674 |
| N2 [min] | 200.333 | 44.667 | 206.571 | 65.912 | 20 | 0.590 | 0.674 |
| N3 [min] | 163.190 | 55.214 | 128.857 | 55.156 | 20 | −4.047 | 0.004 |
| REMS [min] | 56.524 | 34.554 | 55.857 | 32.085 | 20 | −0.085 | 0.933 |

Nap sleep

| | | | | | | | |
|---|---|---|---|---|---|---|---|
| Wake [min] | 13.082 | 12.225 | 25.041 | 17.018 | 21 | 3.727 | 0.005 |
| SOL [min] | 12.873 | 12.098 | 23.818 | 17.220 | 21 | 3.165 | 0.009 |
| N1 [min] | 3.032 | 1.588 | 2.368 | 1.910 | 21 | −1.407 | 0.261 |
| N2 [min] | 20.227 | 9.095 | 11.414 | 10.417 | 21 | −3.608 | 0.005 |
| N3 [min] | 3.345 | 6.320 | 2.309 | 4.303 | 21 | −0.925 | 0.438 |
| REMS [min] | 4.923 | 8.267 | 3.964 | 6.318 | 21 | −0.633 | 0.534 |

Pre-awakening sleep period

| | | | | | | | |
|---|---|---|---|---|---|---|---|
| Wake [min] | 1.568 | 2.417 | 1.955 | 2.890 | 21 | 0.878 | 0.650 |
| N1 [min] | 0.432 | 0.623 | 0.409 | 0.811 | 21 | −0.129 | 0.898 |
| N2 [min] | 4.568 | 3.364 | 5.000 | 3.988 | 21 | 0.480 | 0.795 |
| N3 [min] | 1.341 | 2.427 | 1.886 | 2.899 | 21 | 0.954 | 0.650 |
| REMS [min] | 2.114 | 3.214 | 0.773 | 1.709 | 21 | −1.693 | 0.526 |

SD, standard deviation; df, degrees of freedom; BH-p.value, Benjamini-Hochberg corrected p-values

Means and standard deviations (n=22) in the placebo and caffeine nights are reported. For the night-time sleep recording (top), the nap recording (middle) and the pre-awakening sleep period (bottom). The results of BH-corrected posthoc-tests are also reported.

The formulation validation study corroborated the intended release profile, whereas the maximal plasma concentration was reached after 10.5 hours. As expected based on the in vitro development of the engineered caffeine micropellets, the caffeine curve followed a sustained-release profile, with an efficacious plasma concentration range of ˜5-20 μM, as known to the skilled person attained after 7 hours. The investigated formulation ameliorated sleep inertia symptoms on the subsequent morning following only 4 hours of sleep. The quality of the awakening experience was subjectively improved on behavioural, cognitive, emotional, and physical levels, as indicated on all subscales of the sleep inertia questionnaire. Intriguingly, even though the study participants were sleep restricted, many of them reported less difficulty to rise when compared to a normal morning preceding the experiment, particularly in the caffeine condition. In addition, the formulation exhibited mood enhancing properties, as indicated by increased positive and reduced negative ratings on the PANAS shortly upon wakening. Consistent with our hypothesis that pre-awakening caffeine supply may stimulate cortisol secretion and augment the CAR, we found a prolonged cortisol curve upon waking.

### Example 3. In Vitro Release Measurements of Formulations According to Reference Preparative Experiments 1 and 2

The test parameters were simulated considering the gastric and intestinal conditions and the formulation was developed using a pH and time-dependent approach, as described hereinabove. For simulating conditions of the GI tract, dissolution studies were carried out in media with pH 1.2 (HCl 0.1N), pH 6.5, pH 6.8 and pH 7.2 (phosphate buffer) and samples were tested separately at each medium. Dissolution time was 2 h for medium with pH 1.2, 1 h for medium with pH 6.5, 2 h for medium with pH 6.8 and 5 h for the pH 7.2.

In-vitro test parameters are given in Table 19 and are the same as used in Example 1 (In vitro release measurements of formulations according to preparative experiments 2 to 8 and 12).

TABLE 19
In-vitro test parameters.

| Media | pH 1.2 (2 h) - pH 6.5 (1 h) - pH 6.8 (2 h) - pH | |
|---|---|---|
| | 7.2 (5 h) | |
| Time Points | For pH 1.2: 2^{nd} hour | |
| | For pH 6.5: 1^{st} hour | |
| | For pH 6.8: 2^{nd} hour | |
| | For pH 7.2: 1^{st} hour, 2^{nd} hour, | |
| | 4^{th} hour and 5^{th} hour | |
| Surfactant | None | |
| Stirring Speed | 100 | rpm |
| Apparatus | Basket | |
| Media Volume | 900 ± 1 | mL |
| Temperature | 37 ± 0.5° | C. |

In-vitro test results are given in Table 20.

TABLE 20
In vitro release of formulations of preparative reference examples 1 and 2
(Average ± Standard Deviation).

| Example | pH 1.2 | pH 6.5 | pH 6.8 | pH 7.2 | pH 7.2 | pH 7.2 | pH 7.2 |
|---|---|---|---|---|---|---|---|
| Number | (2 hours) | (1 hour) | (2 hours) | (1 hour) | (2 hours) | (4 hours) | (5 hours) |
| Reference | 0.5 ± 0.1 | 1 ± 0.2 | 2 ± 0.2 | 9 ± 1.2 | 21 ± 0.9 | 33 ± 2.4 | 52 ± 1.9 |
| preparative | | | | | | | |
| example 1 | | | | | | | |
| Reference | 3 ± 0.5 | 10 ± 1.1 | 34 ± 2.5 | 83 ± 2.3 | 90 ± 1.9 | 93 ± 2.2 | 94 ± 0.9 |
| preparative | | | | | | | |
| example 2 | | | | | | | |

Further examples and/or embodiments of the present invention are disclosed in the following numbered items.

1. A pulsatile-release formulation comprising a nootropic agent and a release-controlling polymeric system, wherein the release-controlling polymeric system comprises at least one copolymer, wherein the at least one copolymer is a copolymer of at least methacrylic acid and methyl methacrylate.

2. The pulsatile release formulation comprising a nootropic agent and a release-controlling polymeric system of item 1, wherein the release controlling polymeric system comprises:
(a) a copolymer of methacrylic acid and methyl methacrylate in a molar ratio of 1:2.5 to 1:1.5;
(b) a copolymer of methacrylic acid and methyl methacrylate in a molar ratio of 1:1.4 to 1:0.5; and
(c) a copolymer of ethyl acrylate, methyl methacrylate and trimethylammonioethyl methacrylate chloride, wherein ethyl acrylate and methyl methacrylate are present in the molar ratio of 1:2.5 to 1:1.5 and wherein ethyl acrylate and trimethylammonioethyl methacrylate chloride are present in the molar ratio of 1:0.25 to 1:0.05;
wherein (a) and (b) are present in a weight/weight ratio of between 2 to 6, wherein (c) and (b) are present in a weight/weight ratio of between 0.5 to 2, and wherein the nootropic agent's release is controlled by the release-controlling polymeric system.

3. The pulsatile release formulation of item 1 or 2, wherein (a) has a weight average molecular weight of between 120 000 Da and 130 000 Da, and/or wherein (b) has a weight average molecular weight of between 120 000 Da and 130 000 Da, and/or wherein (c) has a weight average molecular weight of between 30 000 Da and 34 000 Da.

4. The pulsatile release formulation of any one of item 1 to 3, wherein (a) has a weight average molecular weight of about 125 000 Da, and/or wherein (b) has a weight average molecular weight of about 125 000 Da, and/or wherein (c) has a weight average molecular weight of about 32 000 Da.

5. The pulsatile release formulation of any one of items 1 to 4, wherein (a) is a copolymer of methacrylic acid and methyl methacrylate in a molar ratio of about 1:2.

6. The pulsatile release formulation of any one of items 1 to 5, wherein (b) is a copolymer of methacrylic acid and methyl methacrylate in a molar ratio of about 1:1.

7. The pulsatile release formulation of any one of items 1 to 6, wherein (a) and (b) are present in a weight/weight ratio of about 4.

8. The pulsatile release formulation of any one of items 1 to 7, wherein the (c) and (b) are present in a weight/weight ratio of 1.0 to 1.5.

9. The pulsatile release formulation of any one of items 1 to 8, wherein (a) is Eudragit® S and/or (b) is Eudragit® L and/or (c) is Eudragit® RS and/or (c) is Eudragit® RL.

10. The pulsatile-release formulation comprising a nootropic agent and a release-controlling polymeric system of item 1, wherein the release-controlling polymeric system comprises a copolymer of methacrylic acid, methyl methacrylate and methyl acrylate,
wherein the molar ratio of methacrylic acid to the sum of methyl methacrylate and methyl acrylate is between 0.05 to 0.1,
wherein the molar ratio of methyl acrylate and methyl methacrylate is from 2.0 to 2.8, and
wherein the nootropic agent's release is controlled by the release controlling polymeric system.

11. The pulsatile-release formulation of item 10, wherein the weight average molecular weight of the copolymer is between 260 000 Da and 300 000 Da.

12. The pulsatile-release formulation of item 10 or 11, wherein the weight average molecular weight of the copolymer is about 280 000 Da.

13. The pulsatile-release formulation of any one of items 10 to 12, wherein the molar ratio of methacrylic acid to the sum of methyl methacrylate and methyl acrylate is about 0.10, and wherein the molar ratio of methyl acrylate and methyl methacrylate is about 2.3.

14. The pulsatile-release formulation of any one of items 10 to 13, wherein the copolymer is Eudraguard® biotic.

15. The pulsatile-release formulation of any one of items 1 to 14, wherein the nootropic agent is an adenosine receptor agonist.

16. The pulsatile-release formulation of item 15, wherein the adenosine receptor agonist is xanthin derivative, in particular theophylline or a pharmaceutically acceptable salt thereof, theobromine or a pharmaceutically acceptable salt thereof, paraxanthine or a pharmaceutically acceptable salt thereof, or caffeine or a pharmaceutically acceptable salt thereof.

17. The pulsatile-release formulation of any one of items 1 to 16, wherein the nootropic agent is caffeine.

18. The pulsatile-release formulation of any one of items 1 to 14, wherein the nootropic agent is a sympathomimetic, in particular ephedrine or a pharmaceutically acceptable salt thereof, amphetamine or a pharmaceutically acceptable salt thereof, phenylephrine or a pharmaceutically acceptable salt thereof, pseudoephedrine or a pharmaceutically acceptable salt thereof, or etilefrine or a pharmaceutically acceptable salt thereof.

19. The pulsatile-release formulation of any one of items 1 to 14, wherein the nootropic agent is piracetam or a pharmaceutically acceptable salt thereof.

20. The pulsatile-release formulation of any one of items 1 to 14, wherein the nootropic agent is phenylpiracetam or a pharmaceutically acceptable salt thereof.

21. The pulsatile-release formulation of any one of items 1 to 14, wherein the nootropic agent is modafinil or a pharmaceutically acceptable salt thereof.

22. The pulsatile-release formulation of any one of items 1 to 14, wherein the nootropic agent is armodafinil or a pharmaceutically acceptable salt thereof.

23. The pulsatile-release formulation of any one of items 1 to 14, wherein the nootropic agent is methylphenidate or a pharmaceutically acceptable salt thereof.

24. The pulsatile-release formulation of any one of items 1 to 14, wherein the nootropic agent is a serotonin and noradrenalin reuptake inhibitor, in particular venlafaxine or a pharmaceutically acceptable salt thereof, desvenlafaxine or a pharmaceutically acceptable salt thereof, or duloxetine or a pharmaceutically acceptable salt thereof, or wherein the nootropic agent is a selective serotonin reuptake inhibitor, in particular fluoxetine or a pharmaceutically acceptable salt thereof, sertraline or a pharmaceutically acceptable salt thereof, paroxetine or a pharmaceutically acceptable salt thereof, citalopram or a pharmaceutically acceptable salt thereof, escitalopram or a pharmaceutically acceptable salt thereof, or fluvoxamine or a pharmaceutically acceptable salt thereof.

25. The pulsatile-release formulation of any one of items 1 to 24, wherein the ratio of the nootropic agent's weight to the total solid content's weight of the pulsatile-release formulation is between 10:1 to 1:100.

26. The pulsatile-release formulation of any one of items 1 to 25, wherein the ratio of the weight of the release-controlling polymeric system to the weight of the rest of the pulsatile-release formulation is between 1:20 to 5:1.

27. The pulsatile-release formulation of any one of items 1 to 26, further comprising a sedative substance.

28. The pulsatile-release formulation of item 27, wherein the sedative substance is melatonin or a pharmaceutically acceptable salt thereof.

29. The pulsatile-release formulation of item 27, wherein the sedative substance is an antihistaminic, in particular diphenhydramine or a pharmaceutically acceptable salt thereof, dimenhydrinate or a pharmaceutically acceptable salt thereof, or dimetindene or a pharmaceutically acceptable salt thereof.

30. The pulsatile-release formulation of item 27, wherein the sedative substance is an antipsychotic, in particular quetiapine or a pharmaceutically acceptable salt thereof, levomepromazine or a pharmaceutically acceptable salt thereof, or olanzapine or a pharmaceutically acceptable salt thereof.

31. The pulsatile-release formulation of item 27, wherein the sedative substance is trazodone or a pharmaceutically acceptable salt thereof.

32. The pulsatile-release formulation of item 27, wherein the sedative substance is mirtazapine or a pharmaceutically acceptable salt thereof.

33. The pulsatile-release formulation of item 27, wherein the sedative substance is a z-drug, in particular zolpidem or a pharmaceutically acceptable salt thereof, zaleplon or a pharmaceutically acceptable salt thereof, or zolpidem or a pharmaceutically acceptable salt thereof.

34. The pulsatile-release formulation of item 27, wherein the sedative substance is a benzodiazepine, in particular alprazolam or a pharmaceutically acceptable salt thereof, chlordiazepoxide or a pharmaceutically acceptable salt thereof, clonazepam or a pharmaceutically acceptable salt thereof, clorazepate or a pharmaceutically acceptable salt thereof, diazepam or a pharmaceutically acceptable salt thereof, estazolam or a pharmaceutically acceptable salt thereof, flurazepam (Dalmane) or a pharmaceutically acceptable salt thereof, lorazepam or a pharmaceutically acceptable salt thereof, midazolam or a pharmaceutically acceptable salt thereof, oxazepam or a pharmaceutically acceptable salt thereof, temazepam or a pharmaceutically acceptable salt thereof, triazolam or a pharmaceutically acceptable salt thereof, or quazepam or a pharmaceutically acceptable salt thereof.

35. The pulsatile-release formulation of item 27, wherein the sedative substance is doxylamine or a pharmaceutically acceptable salt thereof.

36. The pulsatile-release formulation of item 27, wherein the sedative substance is chlorphenamine or a pharmaceutically acceptable salt thereof.

37. The pulsatile-release formulation of item 27, wherein the sedative substance comprises a phytochemical selected from Valeriana officinali, Humulus lupulus, Lavandula officinalis, Hypericum perforatum, Petasites hybridus, Melissa officinalis, Passiflora incarnata, and Choisya ternata.

38. The pulsatile-release formulation of item 27, wherein the sedative substance comprises an amino acid selected from L-tryptophan, L-theanine, and glycine.

39. The pulsatile-release formulation of item 27, wherein the sedative substance is niacin or a pharmaceutically acceptable salt thereof.

40. The pulsatile-release formulation of item 27, wherein the sedative substance comprises magnesium salt.

41. The pulsatile-release formulation of any one of items 1 to 40 for use in therapy.

42. The pulsatile-release formulation of any one of items 1 to 40 for use in the therapy of morning depression and/or wake-up difficulties in Delayed Sleep Phase Syndrome, Seasonal Affective Disorders, Narcolepsy, Sleep Deprivation, Major Depression, Idiopathic Insomnia, or Sedative-induced Hang-overs.

43. The pulsatile-release formulation for use of item 41 or 42, wherein the nootropic agent is caffeine, and wherein the strength of caffeine per dosage unit is between 10 mg and 1000 mg, more preferably between 40 mg and 320 mg, most preferably 80 mg or 160 mg.

44. The pulsatile-release formulation for use of any one of items 41 to 43, that upon oral administration at bedtime has the ability to decrease subjective and objective signs of sleep inertia immediately upon morning awakening, indicated by improved scores in the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI).

45. The pulsatile-release formulation for use of any one of items 41 to 44, characterized by an in vitro release profile wherein not more than 25% of a nootropic agent is released during the first 5 hours and not less than 75% of a nootropic agent is released until the 9^{th} hour, as measured by the analysis of cumulative dissolved amount of the nootropic agent upon incubation in the dissolution media having a pH of 1.2 for 2 hours, having a pH of 6.5 for 1 hour, having a pH of 6.8 for 2 hours and having a pH of 7.2 for 5 hours.

46. The pulsatile-release formulation for use of any one of items 41 to 45, characterized by an in vivo release profile in human subjects wherein the blood plasma concentration of caffeine reaches 5 μM not earlier than 4 hours following the administration of said pulsatile-release formulation.

47. Use of the pulsatile-release formulation of any one of items 1 to 34 for targeted shifting of the circadian sleep-wake rhythm.

48. The use of item 47, wherein targeted shifting of the circadian sleep-wake rhythm is performed in the case of Jet Lag or Shift Work.

49. The use of item 47 or 48, wherein the nootropic agent is caffeine, and wherein the dose of caffeine is between 10 mg and 1000 mg, more preferably between 40 mg and 320 mg, most preferably 80 mg or 160 mg.

Further examples and/or embodiments of the invention are disclosed in the following numbered paragraphs:

1. A pulsatile-release formulation comprising a nootropic agent and a release-controlling polymeric system, wherein the release-controlling polymeric system comprises at least one copolymer, wherein the at least one copolymer is a copolymer of at least methacrylic acid and methyl methacrylate.

2. The pulsatile release formulation comprising a nootropic agent and a release-controlling polymeric system of paragraph 1, wherein the release controlling polymeric system comprises:
(a) a copolymer of methacrylic acid and methyl methacrylate in a molar ratio of 1:2.5 to 1:1.5;
(b) a copolymer of methacrylic acid and methyl methacrylate in a molar ratio of 1:1.4 to 1:0.5; and
(c) a copolymer of ethyl acrylate, methyl methacrylate and trimethylammonioethyl methacrylate chloride, wherein ethyl acrylate and methyl methacrylate are present in the molar ratio of 1:2.5 to 1:1.5 and wherein ethyl acrylate and trimethylammonioethyl methacrylate chloride are present in the molar ratio of 1:0.25 to 1:0.05;
wherein (a) and (b) are present in a weight/weight ratio of between 2 to 6, wherein (c) and (b) are present in a weight/weight ratio of between 0.5 to 2, and wherein the nootropic agent's release is controlled by the release-controlling polymeric system.

3. The pulsatile release formulation of paragraph 1 or 2, wherein (a) has a weight average molecular weight of between 120 000 Da and 130 000 Da, and/or wherein (b) has a weight average molecular weight of between 120 000 Da and 130 000 Da, and/or wherein (c) has a weight average molecular weight of between 30 000 Da and 34 000 Da, preferably wherein (a) has a weight average molecular weight of about 125 000 Da, and/or wherein (b) has a weight average molecular weight of about 125 000 Da, and/or wherein (c) has a weight average molecular weight of about 32 000 Da.

4. The pulsatile release formulation of any one of paragraphs 1 to 3, wherein (a) is a copolymer of methacrylic acid and methyl methacrylate in a molar ratio of about 1:2,
and/or
wherein (b) is a copolymer of methacrylic acid and methyl methacrylate in a molar ratio of about 1:1,
and/or
wherein (a) and (b) are present in a weight/weight ratio of about 4,
and/or
wherein the (c) and (b) are present in a weight/weight ratio of 1.0 to 1.5.

5. The pulsatile release formulation of any one of paragraphs 1 to 4, wherein (a) is Eudragit® S and/or (b) is Eudragit® L and/or (c) is Eudragit® RS and/or (c) is Eudragit® RL.

6. The pulsatile-release formulation comprising a nootropic agent and a release-controlling polymeric system of paragraph 1, wherein the release-controlling polymeric system comprises a copolymer of methacrylic acid, methyl methacrylate and methyl acrylate,
wherein the molar ratio of methacrylic acid to the sum of methyl methacrylate and methyl acrylate is between 0.05 to 0.15,
wherein the molar ratio of methyl acrylate and methyl methacrylate is from 2.0 to 2.8, and
wherein the nootropic agent's release is controlled by the release controlling polymeric system.

7. The pulsatile-release formulation of paragraph 6, wherein the weight average molecular weight of the copolymer is between 260 000 Da and 300 000 Da, preferably wherein the weight average molecular weight of the copolymer is about 280 000 Da.

8. The pulsatile-release formulation of paragraph 6 or 7, wherein the molar ratio of methacrylic acid to the sum of methyl methacrylate and methyl acrylate is about 0.10, and wherein the molar ratio of methyl acrylate and methyl methacrylate is about 2.3.

9. The pulsatile-release formulation of any one of paragraphs 6 to 8, wherein the copolymer is Eudraguard® biotic.

10. The pulsatile-release formulation of any one of paragraphs 1 to 9, wherein the nootropic agent is an adenosine receptor agonist, preferably wherein the adenosine receptor agonist is xanthin derivative, in particular theophylline or a pharmaceutically acceptable salt thereof, theobromine or a pharmaceutically acceptable salt thereof, paraxanthine or a pharmaceutically acceptable salt thereof, or caffeine or a pharmaceutically acceptable salt thereof.

11. The pulsatile-release formulation of any one of paragraphs 1 to 10, wherein the nootropic agent is caffeine.

12. The pulsatile-release formulation of any one of paragraphs 1 to 11, further comprising a sedative substance, preferably wherein the sedative substance is melatonin or a pharmaceutically acceptable salt thereof.

13. The pulsatile-release formulation of any one of paragraphs 1 to 12 for use in therapy, in particular in the therapy of morning depression and/or wake-up difficulties in Delayed Sleep Phase Syndrome, Seasonal Affective Disorders, Narcolepsy, Sleep Deprivation, Major Depression, Idiopathic Insomnia, or Sedative-induced Hang-overs.

14. The pulsatile-release formulation for use of paragraph 13, wherein the nootropic agent is caffeine, and wherein the strength of caffeine per dosage unit is between 10 mg and 1000 mg, more preferably between 40 mg and 320 mg, most preferably 80 mg or 160 mg, and/or
wherein upon oral administration at bedtime the pulsatile release formulation has the ability to decrease subjective and objective signs of sleep inertia immediately upon morning awakening, indicated by improved scores in the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI), and/or
wherein the pulsatile-release formulation is characterized by an in vitro release profile wherein not more than 25% of a nootropic agent is released during the first 5 hours and not less than 75% of a nootropic agent is released until the 9^{th} hour, as measured by the analysis of cumulative dissolved amount of the nootropic agent upon incubation in the dissolution media having a pH of 1.2 for 2 hours, having a pH of 6.5 for 1 hour, having a pH of 6.8 for 2 hours and having a pH of 7.2 for 5 hours, and/or
wherein the pulsatile-release formulation is characterized by an in vivo release profile in human subjects wherein the blood plasma concentration of caffeine reaches 5 μM not earlier than 4 hours following the administration of said pulsatile-release formulation.

15. Use of the pulsatile-release formulation of any one of paragraphs 1 to 12 for targeted shifting of the circadian sleep-wake rhythm,
preferably wherein targeted shifting of the circadian sleep-wake rhythm is performed in the case of Jet Lag or Shift Work,
and/or
preferably wherein the nootropic agent is caffeine, and preferably wherein the dose of caffeine is between 10 mg and 1000 mg, more preferably between 40 mg and 320 mg, most preferably 80 mg or 160 mg.

1.-8. (canceled)

9. A pulsatile-release formulation comprising a nootropic agent and a release-controlling polymeric system, wherein the release-controlling polymeric system comprises a copolymer of methacrylic acid, methyl methacrylate and methyl acrylate,
wherein the molar ratio of methacrylic acid to the sum of methyl methacrylate and methyl acrylate is about 0.10, and wherein the molar ratio of methyl acrylate and methyl methacrylate is about 2.3, and wherein the nootropic agent's release is controlled by the release controlling polymeric system.

10. The pulsatile-release formulation of claim 9, wherein the weight average molecular weight of the copolymer is between 260 000 Da and 300 000 Da, optionally wherein the weight average molecular weight of the copolymer is about 280 000 Da.

11.-12. (canceled)

13. The pulsatile-release formulation of claim 9, wherein the copolymer is Eudraguard® biotic.

14. The pulsatile-release formulation of claim 9, wherein the nootropic agent is an adenosine receptor agonist.

15. The pulsatile-release formulation of claim 14, wherein the adenosine receptor agonist is xanthin derivative, in particular theophylline or a pharmaceutically acceptable salt thereof, theobromine or a pharmaceutically acceptable salt thereof, paraxanthine or a pharmaceutically acceptable salt thereof, or caffeine or a pharmaceutically acceptable salt thereof.

16. The pulsatile-release formulation of claim 15, wherein the nootropic agent is caffeine.

17. The pulsatile-release formulation of claim 9, wherein the nootropic agent is a sympathomimetic, in particular ephedrine or a pharmaceutically acceptable salt thereof, amphetamine or a pharmaceutically acceptable salt thereof, phenylephrine or a pharmaceutically acceptable salt thereof, pseudoephedrine or a pharmaceutically acceptable salt thereof, or etilefrine or a pharmaceutically acceptable salt thereof, or
wherein the nootropic agent is piracetam or a pharmaceutically acceptable salt thereof, or
wherein the nootropic agent is phenylpiracetam or a pharmaceutically acceptable salt thereof, or
wherein the nootropic agent is modafinil or a pharmaceutically acceptable salt thereof, or
wherein the nootropic agent is armodafinil or a pharmaceutically acceptable salt thereof, or
wherein the nootropic agent is methylphenidate or a pharmaceutically acceptable salt thereof, or
wherein the nootropic agent is a serotonin and noradrenalin reuptake inhibitor, in particular venlafaxine or a pharmaceutically acceptable salt thereof, desvenlafaxine or a pharmaceutically acceptable salt thereof, or duloxetine or a pharmaceutically acceptable salt thereof, or wherein the nootropic agent is a selective serotonin reuptake inhibitor, in particular fluoxetine or a pharmaceutically acceptable salt thereof, sertraline or a pharmaceutically acceptable salt thereof, paroxetine or a pharmaceutically acceptable salt thereof, citalopram or a pharmaceutically acceptable salt thereof, escitalopram or a pharmaceutically acceptable salt thereof, or fluvoxamine or a pharmaceutically acceptable salt thereof.

18.-23. (canceled)

24. The pulsatile-release formulation of claim 9, wherein the ratio of the nootropic agent's weight to the total solid content's weight of the pulsatile-release formulation is between 10:1 to 1:100, optionally wherein the ratio of the weight of the release-controlling polymeric system to the weight of the rest of the pulsatile-release formulation is between 1:20 to 5:1.

25. (canceled)

26. The pulsatile-release formulation of claim 9, further comprising a sedative substance.

27. The pulsatile-release formulation of claim 26, wherein the sedative substance is melatonin or a pharmaceutically acceptable salt thereof, or
wherein the sedative substance is an antihistaminic, in particular diphenhydramine or a pharmaceutically acceptable salt thereof, dimenhydrinate or a pharmaceutically acceptable salt thereof, or dimetindene or a pharmaceutically acceptable salt thereof, or
wherein the sedative substance is an antipsychotic, in particular quetiapine or a pharmaceutically acceptable salt thereof, levomepromazine or a pharmaceutically acceptable salt thereof, or olanzapine or a pharmaceutically acceptable salt thereof, or
wherein the sedative substance is trazodone or a pharmaceutically acceptable salt thereof, or
wherein the sedative substance is mirtazapine or a pharmaceutically acceptable salt thereof, or
wherein the sedative substance is a z-drug, in particular zolpidem or a pharmaceutically acceptable salt thereof, zaleplon or a pharmaceutically acceptable salt thereof, or zolpidem or a pharmaceutically acceptable salt thereof, or
wherein the sedative substance is a benzodiazepine, in particular alprazolam or a pharmaceutically acceptable salt thereof, chlordiazepoxide or a pharmaceutically acceptable salt thereof, clonazepam or a pharmaceutically acceptable salt thereof, clorazepate or a pharmaceutically acceptable salt thereof, diazepam or a pharmaceutically acceptable salt thereof, estazolam or a pharmaceutically acceptable salt thereof, flurazepam (Dalmane) or a pharmaceutically acceptable salt thereof, lorazepam or a pharmaceutically acceptable salt thereof, midazolam or a pharmaceutically acceptable salt thereof, oxazepam or a pharmaceutically acceptable salt thereof, temazepam or a pharmaceutically acceptable salt thereof, triazolam or a pharmaceutically acceptable salt thereof, or quazepam or a pharmaceutically acceptable salt thereof, or
wherein the sedative substance is doxylamine or a pharmaceutically acceptable salt thereof, or
wherein the sedative substance is chlorphenamine or a pharmaceutically acceptable salt thereof, or
wherein the sedative substance comprises a phytochemical selected from Valeriana officinali, Humulus lupulus, Lavandula officinalis, Hypericum perforatum, Petasites hybridus, Melissa officinalis, Passiflora incarnata, and Choisya ternata, or
wherein the sedative substance comprises an amino acid selected from L-tryptophan, L-theanine, and glycine, or
wherein the sedative substance is niacin or a pharmaceutically acceptable salt thereof, or
wherein the sedative substance comprises magnesium salt.

28.-40. (canceled)

41. A method for treating morning depression and/or wake-up difficulties in Delayed Sleep Phase Syndrome, Seasonal Affective Disorders, Narcolepsy, Sleep Deprivation, Major Depression, Idiopathic Insomnia, or Sedative-induced Hang-overs in a subject in need thereof, comprising the step of administering an effective amount of the pulsatile release formulation of claim 9 to the subject.

42. The method of claim 41, wherein the nootropic agent is caffeine, and wherein the strength of caffeine per dosage unit is between 10 mg and 1000 mg, optionally between 40 mg and 320 mg, optionally 80 mg or 160 mg.

43. The method of claim 41, wherein the nootropic agent is caffeine, and wherein the strength of caffeine per dosage unit is 60 mg or 160 mg.

44. The method of claim 41, wherein said pulsatile-release formulation upon oral administration at bedtime decreases subjective and objective signs of sleep inertia immediately upon morning awakening, indicated by improved scores in the Sleep Inertia Questionnaire and the Positive and Negative Affective Schedule (PANAS), increased performance in the psychomotor vigilance task (PVT) enhanced Cortisol Awakening Response (CAR), improved ratings in the Montgomery-Asberg Depression Rating Scale (MADRS), Hamilton Depression Rating Scale (HDRS) and/or Beck Depression Inventory (BDI).

45. The method of claim 41, wherein said pulsatile-release formulation is characterized by an in vitro release profile wherein not more than 25% of a nootropic agent is released during the first 5 hours and not less than 75% of a nootropic agent is released until the 9^{th} hour, as measured by the analysis of cumulative dissolved amount of the nootropic agent upon incubation in the dissolution media having a pH of 1.2 for 2 hours, having a pH of 6.5 for 1 hour, having a pH of 6.8 for 2 hours and having a pH of 7.2 for 5 hours.

46. The method of claim 41, wherein said pulsatile-release formulation, is characterized by an in vivo release profile in human subjects wherein the blood plasma concentration of caffeine reaches 5 μM not earlier than 4 hours following the administration of said pulsatile-release formulation.

47. A method for targeted shifting of the circadian sleep-wake rhythm in a subject comprising administering the pulsatile release formulation of claim 9 to the subject.

48. The method of claim 47, wherein targeted shifting of the circadian sleep-wake rhythm is performed in the case of Jet Lag or Shift Work.

49. The method of claim 47, wherein the nootropic agent is caffeine, and wherein the dose of caffeine is between 10 mg and 1000 mg, optionally between 40 mg and 320 mg, optionally 80 mg or 160 mg.

50. The method of claim 47, wherein the nootropic agent is caffeine, and wherein the strength of caffeine per dosage unit is 60 mg or 160 mg.
